(19) United States
(12) Patent Application Publication
(10) Pub. No.: US 2023/0310514 A1
(43) Pub. Date: Oct. 5, 2023

(54) Dietary Method for Treating Arthritis

(71) Applicant: Valeriy Sibilkov, Whitefish Bay, WI (US)

(72) Inventor: Valeriy Sibilkov, Whitefish Bay, WI (US)

(21) Appl. No.: 17/674,721

(22) Filed: Feb. 17, 2022

(51) Int. Cl. A61K 35/74; A61K 36/06; A61P 19/02

(52) U.S. Cl. CPC A61K 35/74; A61K 36/06; A61P 19/02

(57) ABSTRACT

The inventive method treats arthritis for a person by reducing consumption of purposely fermented or acidified foods. The method includes the steps of identifying offending sources of nutrition including purposely fermented or purposefully acidified foods, ranking those foods from lower to higher in acidity or fermentation, determining pre-treatment consumption amounts and a base level of consumed offending sources of nutrition, and reducing consumption of the offending sources a sufficient amount to alleviate pain and discomfort for the person. Once the base level is incrementally reduced to about 50% of the pre-treatment base level, arthritic pain and symptoms are monitored. If necessary, the base level is further incrementally reduced to about 35%, 20%, 10% or 5% of the pre-treatment base level until pain and symptoms subside.

### TECHNICAL FIELD OF THE INVENTION

This invention relates to a dietary method for treating arthritis by identifying purposefully fermented and acidic foods and beverages consumed, determining a pre-treatment weighted average base level of offending sources of nutrition based on a list of foods, beverages and sources of nutrition that includes their fermentation and acidity ranking, and reducing consumption of the offending sources to below a predetermined percentage.

### BACKGROUND OF THE INVENTION

The idea that diet is related to illness has been known since ancient times. People attempted to avoid illness and death by manipulating the foods they ate. The strategy of controlling diet worked for foods containing substances that cause immediate death. People learned that eating certain mushrooms makes them feel ill or even causes death. Similarly, consumption of hemlock (Cicuta virosa) may result in immediate adverse health consequences, as can the consumption of the liver of a polar bear or a seal. Humans have known for centuries that eating carelessly prepared fugu (puffer fish) can cause death within a few hours. We recently learned this is caused by the neurotoxin tetrodotoxin, which is not produced by the fugu, but by certain bacteria that infect them. This knowledge was by and large obtained through empirical observation. Some poor souls ate the fish and died shortly thereafter, while others who did not eat the fish were fine. The ancient Japanese were able to figure out that certain fish and illness or death are related. And, in order to avoid illness or death, they had to avoid fugu.

Developed countries have recently seen a rise in certain aliments and diseases. These “diseases of affluence” include heart diseases, different types of oncological ailments, diabetes, various forms of arthritis, allergies, asthma and various autoimmune diseases. Scientists have been trying to establish the causes of the unfortunate upward trend in these diseases and to find treatments for them. Yet, identifying substances that do not produce immediate health effects, but rather lead to a gradual decline in health and the lengthy development of disease is difficult. This is due to the lag between the person’s contact with or consumption of the substance and the observation of the symptoms the substance causes. Identifying foods and beverages that slowly deteriorate health is also difficult because a multitude of environmental, genetic, nutritional, behavioral, social and economic factors affect a person. Identification of these offending substances needs careful analysis and understanding of existing knowledge and possibly new evidence.

Of the various fields of literature dealing with the effects of food on human health, two fields of literature are notable. One field of literature studies histamine and the other studies acid-alkaline balance. Histamine is a substance that regulates various functions in the human body, including the immune system. The literature studying histamine notes that human bodies naturally produce a certain level of histamine for biological functions and store some of it. The literature also notes that certain foods and beverages contain histamine. When such foods are ingested, the body may have to neutralize this external histamine in order to control internal histamine levels. Yet, a subset of the general population cannot sufficiently process and neutralize external histamine. Upon consuming and digesting histamine-containing foods and beverages, such people may experience a rise in the levels of histamine, which can lead to abnormal manifestations and reactions in various organs. The literature terms the condition of inadequate histamine processing as “histamine intolerance,” and diagnoses the related manifestations as such. Apparently, less than one percent (1%) of the population is affected (Laura Maintz and Natalija Novak, 2007, Histamine and histamine intolerance, The American Journal of Clinical Nutrition; vol. 85, pp. 1185-1196). To treat histamine intolerance, patients are advised to follow a “histamine-free diet,” which excludes foods that contain histamine, and foods that can cause the release of internally-stored histamine. Researchers find that such steps lead to a reduction in symptoms in people diagnosed as histamine intolerant. For example, referenced in Laura Maintz and Natalija Novak (Maintz, L., Novak, N., 2007, Histamine and histamine intolerance, The American Journal of Clinical Nutrition; vol. 85, pp. 1185-1196) studies (Steinbrecher I, Jarisch R., 2005, Histamin und Kopfschmerz. Allergologie, vol. 28, pp. 84-91; Wantke F, Gotz M, Jarisch R., 1993, Histamine-free diet: treatment of choice for histamine-induced food intolerance and supporting treatment for chronic headaches. Clin Exp Allergy, vol. 23, pp. 982-985) report evidence consistent with the disappearance of histamine-related symptoms when following a histamine-avoidance diet by patients diagnosed as “histamine intolerant.”

The literature that studies acid-alkaline balance subdivides foods into “acid-forming” and “alkaline-forming,” usually based on the chemistry of the ash that results upon burning foods or, alternatively, based on the Potential Renal Acid Load (or PRAL) formula (see, Remer, T., Manz, F., 1994, Estimation of the renal net acid excretion by adults consuming diets containing variable amounts of protein, The American Journal of Clinical Nutrition, vol. 59, pp. 1356-1361, Remer, T., Manz, F., 1995, Potential Renal Acid load of foods and its influence on urine pH, Journal of the American Dietetic Association, vol. 95(7), pp. 791-797). The literature argues that consuming respective foods changes the balance of the body and blood towards acidic or alkaline, and significant imbalance in either direction leads to various conditions and diseases, including autoimmune diseases, allergies, asthma, arthritis, heart diseases, and oncological diseases. This argument is known as the “acid-ash hypothesis.” It is argued that since acid-forming foods dominate the modern diet, the imbalance is mainly on the acidic side. Therefore, it is argued, acid-forming foods lead to diseases, and one needs to balance acid-forming foods with alkaline-forming foods, as well as avoid certain acid-forming foods. The classification of foods proposed by the literature is often not intuitive. For example, lemons, which appear to be quite acidic in nature, are classified as “alkaline-forming.” And most grains and pasta are classified as “acid-forming.” Thus, according to this classification, one way to balance the effects of eating pasta is to eat lemons.

The argument against the acid-ash hypothesis is that a well-functioning body can regulate the acid-alkaline balance within a narrow range, and has mechanisms to cope with an imbalance. It is similar to the ability to maintain body temperature around 36.6° C. (98° F.), as the body can warm itself up by burning nutrients, and cool itself down via perspiration. The scientific evidence on the acid-ash hypothesis is mixed. Some studies fail to find broad empirical support for the idea that the acid-alkaline attributes of foods matter, while other studies report evidence that some minerals that are alkaline (i.e., calcium) lead to improvements in certain symptoms. Gerry K. Schwalfenberg’s 2012 meta-study (Schwalfenberg, G.K., 2012, The Alkaline Diet: Is There Evidence That an Alkaline pH Diet Benefits Health?, Journal of Environmental and Public Health, vol. 2012, pp. 1-7) of the acid-ash hypothesis reports some evidence consistent with the idea that certain alkaline mineral supplementation provide health benefits (Barzel, U. S., Massey, L. K., 1998, Excess dietary protein can adversely affect bone, Journal of Nutrition, vol. 128(6), pp. 1051-1053; J. Vormann, J., Worlitschek, M., Goedecke, T., Silver, B., 2001, Supplementation with alkaline minerals reduces symptoms in patients with chronic low back pain, Journal of Trace Elements in Medicine and Biology, vol. 15(2-3), pp. 179-183; Schwalfenberg, G. K., 2009, Improvement of chronic back pain or failed back surgery with vitamin D repletion: a case series, Journal of the American Board of Family Medicine, vol. 22, no.1, pp. 69-74). However, the same meta-study cites studies that find no significant correlation between acid load and various health metrics (Supplee, J. D., Duncan, G. E., Bruemmer, B., Goldberg, J., Wen, Y., Henderson, J. A., 2011, Soda intake and osteoporosis risk in postmenopausal American-Indian women, Public Health Nutrition, pp. 1-7). Professor Schwalfenberg concludes that “At this time, there are limited scientific studies in this area [ ]” (page 5 of Schwalfenberg, G.K., 2012, The Alkaline Diet: Is There Evidence That an Alkaline pH Diet Benefits Health?, Journal of Environmental and Public Health, vol. 2012, pp. 1-7).

There are several problems with the histamine and the acid-alkaline balance literatures. First, with respect to the acid-alkaline balance literature or the acid-ash hypothesis, the first problem is their classification of foods. The literature requires classifying the universe of foods and beverages into acid-forming and alkaline-forming based on the characteristics of the ash that results upon burning foods or the PRAL score. The approach makes a strong implicit assumption that digestion is comparable to burning foods. Furthermore, multiple versions of classifications exist, which disagree on classifications of some foods. Also, classifications per se are often unintuitive and overly complex.

Second, the focus of the literature - manifested even in its name - is on the need to balance acid-forming foods with alkaline-forming. The implicit - but yet to be verified -assumption is that alkaline-forming foods have sufficient potential to balance acid-forming foods. It is also possible that, as the opponents of the theory argue, the human body can cope with most common acid-forming foods even without the help of alkaline-forming foods.

Perhaps as a result of the first two problems is a third problem. The empirical evidence on the effectiveness and usefulness of the existing approaches and food classifications is limited and mixed. Existing research fails to find broad empirical support for the idea that acid-alkaline attributes of foods matter for health outcomes, including outcomes such as arthritis and other skeletal and joint pains and conditions. Additionally, as discussed below, some empirical evidence conflicts with the arguments and predictions of the acid-ash hypothesis.

Which brings us to a fourth problem. The literature’s handling of vegetables is suspect and the empirical evidence appears inconsistent. As already noted, the literature argues for the need to balance acid-forming foods with alkaline-forming foods comprised mostly of vegetables. Under the argument, a negative relation between vegetables supply and disease prevalence should be observed. That is, a negative and significant coefficient of vegetables in a regression of disease prevalence should be seen. Yet, no such relation is observed. (See Tables 4 and 5 below). The coefficients of vegetables are positive and statistically significant in three out of five models (Table 4, Models 8-10, 0.460, 7.726 and 3.992, respectively). None of the coefficients for vegetables are negative and statistically significant. Vegetables have a positive - not negative - correlation with the prevalence of arthritis. This directly contradicts the acidity balance literature.

A fifth problem is that the handling of fruits is also suspect. The literature identifies fruit as mostly acid-forming, with the exception of some citrus fruit, such as lemons and limes, which the literature argues have an alkaline effect on the body. Under the argument, we expect to observe a positive relation between the supply of fruit that are not citrus (which include apples, grapes, bananas, melons, and others) and disease prevalence, and a negative relation between the supply of citrus fruit (which include lemons, limes, oranges, mandarins, and grapefruit) and disease prevalence.

Again, the empirical evidence provides little support for these predictions. In a regression of arthritis prevalence on the supply of different types of foods, the coefficients of the supply of fruit that are not citrus are all negative (Table 5, Models 11-15, -4.899, -0.061, -1.034, -9.325 and -3.675, respectively) and four out of five are statistically significant. Furthermore, four of five coefficients of citrus fruit are negative (Table 5, Models 11 and 13-15, -1.017, -0.511, -10.019 and -0.086, respectively), and only one positive (0.409 in Model 12). (See Table 5 below). Importantly, none of coefficients of citrus fruit is statistically significant. This is inconsistent with the acidity balance literature about the effect of fruit on disease. Specifically, it appears that higher supply of fruit that are not citrus is associated with the reduction in arthritis prevalence, but the same is not the case for the supply of citrus fruit. That particular regression also reveals the limitations of the general notion underlying the acidity balance literature in its current form. Specifically, judging by the correlations between the supply of various food categories and arthritis prevalence as discussed below (See Table 5), none of the foods categories appear to have a negative relation with arthritis that is similar in magnitude to the positive relation between arthritis and wine. Put differently, the result does not support the idea that adverse effects of certain foods (such as wine) are reasonably offset by the consumption of other foods, which appears to be the idea that is central to the acidity-balance literature.

In turn, the histamine intolerance literature suffers from at least the following problems. There is evidence that the histamine content of foods may not be the culprit, or may not be the only culprit. First, some foods that generally fall under the histamine-rich profile -such as wine - lead to symptoms even if these foods have low histamine content. For instance, some low histamine wine apparently causes similar symptoms as high histamine wine. Second, some foods that do not fall under the histamine-rich profile and have no or low histamine cause symptoms similar to symptoms observed with histamine-rich foods. These foods are classified as “histamine liberators”, and the literature argues that such foods cause the release of the internal histamine stored by the body. Third, there is evidence that ingesting some foods classified as histamine-rich can cause symptoms even in healthy adults that seemingly should have no difficulty processing histamine. This gives rise to the idea that even though the literature denotes histamine intolerance as an abnormal condition that affects only a small subset of the population, it is possible that foods classified as histamine-rich are generally problematic.

Another problem with the histamine intolerance literature is that it typically focuses on immediately observable manifestations and may omit long-term effects. However, the absence of observable manifestations after ingesting certain foods in a more general population is not necessarily evidence that there is no adverse effect, which can include various forms of arthritis and other skeletal and joint pains and conditions. It is possible that certain foods identified as histamine-rich can cause systemic dysfunction even in the general population.

A further problem with the studies of histamine and acid-alkaline balance hypothesis is that they fail to properly recognize and identify the causal relationship between acidified foods, fermented foods, and systemic conditions, such as arthritis.

The present invention is intended to solve these and other problems and inconsistencies.

### BRIEF DESCRIPTION OF THE INVENTION

The present invention relates to a dietary method of treating arthritis for a person by reducing or eliminating the consumption of purposely fermented and acidified foods. The method includes the steps of identifying foods consumed by the person that are purposefully fermented or purposefully acidified, designating a strength factor for those foods based on acidity or fermentation, determining a pre-treatment base level consumption of purposefully fermented and purposely acidified sources of nutrition for the person, and reducing consumption of the purposefully fermented and acidified sources for the person to less than fifty percent (50%) of the person’s pre-treatment base level. The method further includes the steps of monitoring the pain and symptoms for the treated person, and if necessary, further incrementally reducing the consumption of purposefully fermented and acidified sources to about thirty-five percent (35%), twenty percent (20%), ten percent (10%) or five percent (5%) of the pre-treatment base level until his or her pain and symptoms subside.

One advantage of the present dietary method for treating arthritis is that it identifies and organizes meaningful (offending) and non-meaningful (non-offending) foods and food groups for the treatment of arthritis. The confusion in categorizing foods based on histamine content or acid-ash content is eliminated. The present method of treating arthritis by reducing fermented and acidic foods simplifies the organization of foods into meaningful and non-meaningful groups.

Another advantage of the present dietary method of treating arthritis is it provides a reliable approach to determine, calculate or otherwise gauge the base level or load of offending sources of nutrition consumed by a person during a consumption time period. First, a pre-treatment base level of offending sources is determined. Modifications to the person’s diet are then made to develop a treatment base level. All or some offending sources of nutrition are eliminated or replaced with less offending sources of nutrition. The pre-treatment base level is then compared to the treatment base level, which allows a person to determine or calculate a desired percentage drop in his or her base level of offending sources of nutrition.

A further advantage of the present dietary method of treating arthritis is its adjustability to meet the need of the specific person being treated without drastically altering his or her daily diet. A person can replace some offending sources of nutrition with non-offending or less offending sources of nutrition to the degree necessary to treat his or her arthritis, while maintaining as much of his or her normal diet as possible. While eliminating all offending sources of nutrition is certainly possible using this method, the method provides the advantage of allowing a person to reduce his or her base level or load of offending sources of nutrition to the degree necessary to treat his or her arthritis. Drastic dietary changes and “one size fits all” dietary changes that would otherwise discourage dietary treatment are avoided.

A still further advantage of the present dietary method of treating arthritis is its gradual easing of a person into dietary changes. The person can start with more obvious dietary changes, such as eliminating the most egregious offending sources of nutrition or replacing them with alternate less offending sources of nutrition to drop his or her pre-treatment base level to a first treatment base level or tier. Then, if these more obvious dietary changes are insufficient to reduce arthritic pain to a tolerable level, the person can implement further dietary changes to drop his or her base level to a second lower treatment base level or tier. This gradual easing of changes can be repeated several times for the person to encourage dietary treatment, without the need for immediate drastic dietary changes that many people find intolerable.

A still further advantage of the dietary method for treating arthritis is a reduction in pain associated with existing arthritis and an increase in pain free mobility for a person. This reduction in pain and increased mobility leads to a generally healthier lifestyle, reduced dependency on pain killers, reduction in the need for outside care, and an overall higher quality of life.

A still further advantage of the dietary method for treating arthritis is it reduces the likelihood of an increase in future pain, damage and other manifestations associated with existing arthritis. With respect to the possible development of arthritis in people without severe or any arthritis, the reduced consumption of acidic and fermented foods reduces the likelihood of developing pain, skeletal and joint damage, and other manifestations associated with arthritis in the future.

A still further advantage of the dietary method for treating arthritis is it reduces skeletal and joint damage, and other manifestations, as well as other systemic conditions, such as osteoarthritis, rheumatoid arthritis, gout, low back pain, and neck pain.

Other aspects and advantages of the invention will become apparent upon making reference to the specification, claims and drawings.

### BRIEF DESCRIPTION OF THE DRAWINGS

FIG. 1 is a schematic drawing showing the steps in a first, aggressive or hard-line strategy [10, 170] embodiment of the present method for treating arthritis.

FIG. 2 is a schematic drawing showing the steps in a second, tiered or gradual strategy [10, 180] embodiment of the present method for treating arthritis.

### DESCRIPTION OF THE PREFERRED EMBODIMENTS

While this invention is susceptible to embodiment in many different forms, the drawings show and the specification describes in detail two preferred embodiments of the invention. It should be understood that the drawings and specification are to be considered an exemplification of the principles of the invention. They are not intended to limit the broad aspects of the invention to the embodiments illustrated.

The spoilage of food and beverages is caused by the consumption of organic matter by microorganisms, namely bacteria and fungi. People ferment foods and beverages, such as cheese and wine, and add acids to foods, such as pickles and soft drinks, so they do not spoil. In other words, we ferment and add acids to foods and beverages so that other living organisms do not consume them. While these foods become unfit for consumption by other organisms - presumably because it causes critical harm to those organisms - these preserved foods cause similar harm to humans. But humans are generally made up of greater mass of cells. The harm of purposefully fermented and acid additives do not manifest immediately, and the relation between the cause and the harm is overlooked.

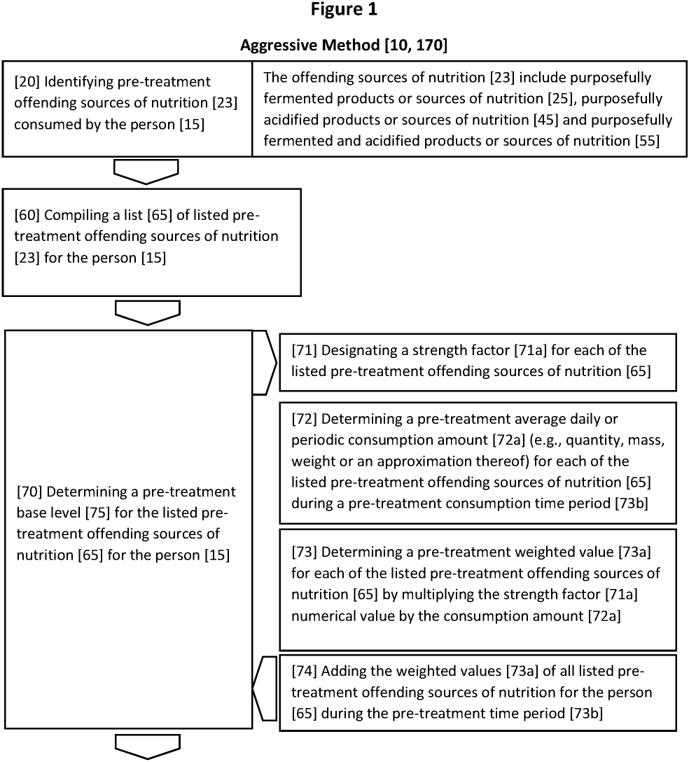

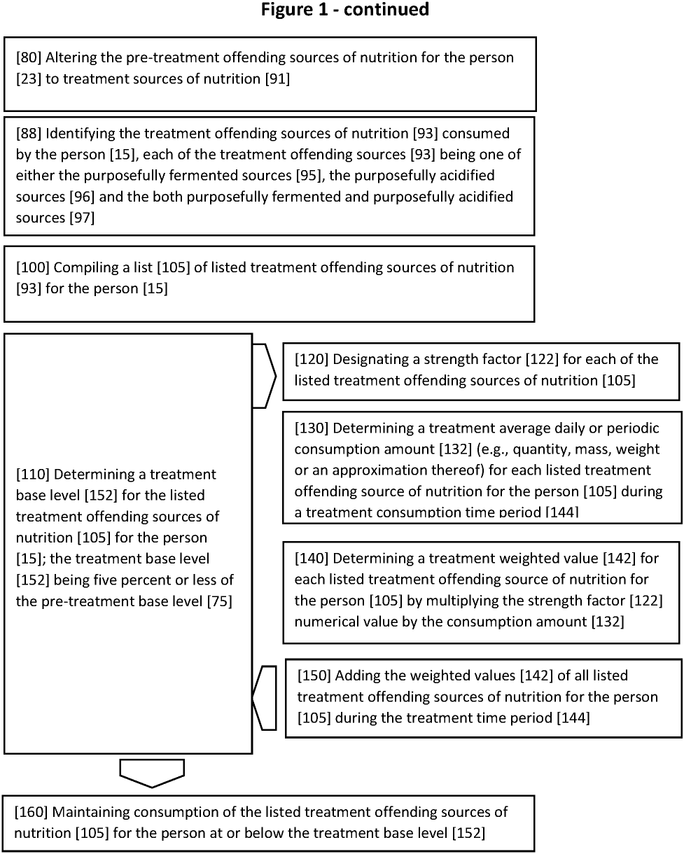

The present method for treating arthritis [10] and associated diseases in a person [15], is to reduce or avoid consumption of offending foods or offending sources of nutrition [23]. These offending sources of nutrition [23] include purposefully fermented foods [25], foods that have been processed by the addition of acids (purposefully acidified) [45], and foods that are acids [47] themselves. The person [15] refers to whomever the method is directed or applied towards (FIG. 1).

One step in the present method of treating arthritis [10] is identifying [20] the pre-treatment offending sources of nutrition [23] consumed by the person [15]. The step [20] involves the identification of all pre-treatment foods or sources of nutrition that the person consumes [21] in a pre-treatment consumption time period [73b], including the identification of pre-treatment offending sources of nutrition [23] and the identification of pre-treatment non-offending sources of nutrition [22]. The offending foods or sources of nutrition [23] include purposefully fermented foods or sources of nutrition [25], purposefully acidified foods or sources of nutrition [45] and purposefully fermented and acidified foods or sources of nutrition [55]. Table A provides a summary of the identification of offending sources of nutrition [23]. Correspondingly, non-offending sources of nutrition [22] are foods or sources of nutrition that have not been identified as offending [23].

TABLE A

Pre-treatment and treatment offending sources of nutrition [23/93]

| | | |
|---|---|---|
| Classification | Attribute | Examples |
| Purposefully fermented sources of nutrition [25/95] | - Deliberately fermented, one hour (cultured microorganisms (fermenting agent [40]), yeast (Kingdom of Fungi [42]) or bacteria (bacteria domain [41])) [25a] - Deliberately fermented, one day (wild microorganisms (fermenting agent [40]), yeast (Kingdom of Fungi [42]) or bacteria (bacteria domain [41])) [25b] | Purposefully fermented groups [30]: Fermented bean foods group [31] Fermented grain foods group [32] Fermented vegetable foods group [33] Fermented fruit foods group [34] Fermented honey foods group [35] Fermented dairy foods group [36] Fermented fish foods group [37] Fermented meat foods group [38] Fermented tea foods group [39] |
| Purposefully acidified sources of nutrition [45/96] | - Deliberately acidified using an acid [45a] - Acids group [47] with pH below about 4 [47a] - Naturally acidic sources [48] | Deliberately acidified using an acid [45a]: foods with citric acid added, foods with fumaric acid added, foods with lactic acid added, foods with acetic acid added, foods with ascorbic acid added, foods with folic acid added, foods with malic acid added, foods with sorbic acid added, foods with erythorbic acid added, foods with stearic acid added, foods with tartaric acid added and foods with phosphoric acid added Example: bottled sweet tea with phosphoric acid added, bottled sweet tea with citric acid added, bottled vitamin water with ascorbic acid and citric acid added, chicken sausages with vinegar added, salmon caviar with sorbic acid added Acids group [47] with pH below about 4 [47a]: Example: white vinegar Naturally acidic sources [48]: Example: apple cider vinegar, balsamic vinegar |
| Purposefully | Satisfies one or more in both: | Example: yogurt with filling or topping with acid |
| fermented and acidified sources of nutrition [55/97] | A) Purposefully fermented sources of nutrition [25/95] and B) Purposefully acidified sources of nutrition [45/96] | added, frozen yogurt with acid added, cheese with ascorbic or citric acid added, bread with acid added, pizza with acid added with cheese with acid added, aged or cured sausage with acid added, bottled kvas with acid added, bottled kombucha with acid added, cheese spread with ascorbic or citric acid added, apple pie with yeast with filling with acid added, vinegar-based dressing with blue cheese |

Foods that are purposefully fermented [25] are foods, beverages and sources of nutrition that wholly or in part have been fermented using cultured microorganisms, cultured yeast or cultured bacteria for more than one hour [25a], or using wild microorganisms, yeast or bacteria for more than one day [25b]. As an example, foods and beverages that can be purposefully fermented include beans, grains, vegetables, fruit, honey, dairy, fish, meat and tea [30]. Bean-based foods [31] that are purposefully fermented [25] include the following group of foods: cheonggukjang, doenjang, fermented bean curd, miso, natto, soy sauce, stinky tofu, tempeh, oncom, soybean paste, Beijing mung bean milk, kinama and iru. Grain-based foods [32] that are purposefully fermented [25] include the following group of foods: batter made from rice and lentil (Vigna mungo), amazake, beer, bread, choujiu, gamju, injera, kvass, makgeolli, murri, ogi, rejuvelac, sake, sikhye, sourdough, sowans, rice wine, malt whisky, grain whisky, idli, dosa, Bangla vodka, boza and chicha. Vegetable-based foods [33] that are purposefully fermented [25] include the following group of foods: kimchi, pickles, sauerkraut, Indian pickle, gundruk and tursu. Fruit-based foods [34] that are purposefully fermented [25] include the following group of foods: wine, vinegar, cider, perry, brandy, atchara, nata de coco, burong mangga, asinan, vişinată, chocolate and rak₁. Honey-based foods [35] that are purposefully fermented [25] include the following group of foods: mead and metheglin. Dairy-based foods [36] that are purposefully fermented [25] include the following group of foods: cheese, kefir, ryazhenka, buttermilk, kumis, shubat, quark, filmjölk, crème fraîche, smetana, skyr and yogurt. Fish-based foods [37] that are purposefully fermented [25] include the following group of foods: bagoong, faseekh, fish sauce, Garum, Hákarl, jeotgal, rakfisk, shrimp paste, surströmming and shidal. Meat-based foods [38] that are purposefully fermented [25] include the following group of foods: chin som mok, chorizo, salami, sucuk, pepperoni, nem chua, som moo and saucisson. Tea-based [39] fermented foods [25] include the following group of foods: pu-erh tea and kombucha.

Fermentation occurs by adding one or more cultured or wild fermenting agents from a group of fermenting agents [40], which include acid-producing microorganisms, bacteria [41] and yeasts [42]. Acid-producing microorganisms include organisms from the Bacteria domain [41], including Firmicutes such as Bacilli, Proteobacteria, Acidobacteria, and microorganisms from the kingdom of Fungi (molds or yeasts) [42]. The most common species of these microorganisms [40] belong to the following genera: Rhizopus, Aspergillus, Mucor, Amylomyces, Endomycopsis, Saccharomyces, Hansenula anomala, Lactobacillus (Lactobacillales order), Acetobacter (Acetobacteraceae family).

Foods or sources of nutrition that are purposefully acidified or acidic [45] are foods, beverages and sources of nutrition that wholly or in part have been modified by adding one or more acids from the group of acids [47] to them, or are acids themselves [48]. This includes foods or sources of nutrition that contain an ingredient which has been modified accordingly. Acids [47a] that are most commonly added to foods or sources of nutrition include citric acid, fumaric acid, lactic acid, acetic acid (vinegar is diluted acetic acid), ascorbic acid (e.g., vitamin C), folic acid (e.g., vitamin B9), malic acid, sorbic acid, erythorbic acid, stearic acid, tartaric acid and phosphoric acid [47a]. Most of these acids [47a] have the constant for dissociation of the first proton (pH) of less than 4. As a result, their [47a] addition reduces the pH level of the foods significantly, often to below 5. As an example, the following foods, beverages and sources of nutrition are purposefully acidified [45]: bottled sweet tea with phosphoric acid added, bottled sweet tea with citric acid added, bottled vitamin water with ascorbic acid and citric acid added, tomato-based pasta sauce with citric acid added, chicken sausages with vinegar added, apple sauce with ascorbic acid added, canned salmon caviar with sorbic acid added, white vinegar, rice vinegar, apple cider vinegar, balsamic vinegar.

Foods or sources of nutrition that are purposefully fermented and acidified [55] are foods and beverages that wholly or in part (that is, some components or ingredients thereof) have been purposefully acidified as described above (have been modified by adding one or more acids [47a] to them, or are acids themselves) and that have been purposefully fermented as described above (have been fermented using cultured microorganisms, cultured yeast or cultured bacteria for more than one hour [25a], or using wild microorganisms, yeast or bacteria for more than one day [25b]). As an example, the following foods, beverages and sources of nutrition are both purposefully fermented and acidified: yogurt with filling or topping with acid added, frozen yogurt with acid added, cheese with ascorbic or citric acid added, bread with acid added, pizza with acid added with cheese with acid added, aged or cured sausage with acid added, bottled kvas with acid added, bottled kombucha with acid added, cheese spread with ascorbic or citric acid added, apple pie with yeast with filling with acid added, vinegar-based dressing with blue cheese.

Another step in the method of treating arthritis [10] is compiling [60] a list [65] of the offending sources of nutrition [23] for the person [15] (FIG. 1). The outcome of this step [60] is a list of pre-treatment offending sources of nutrition for the person [65].

Table B presents an example of the step of identifying the pre-treatment offending sources of nutrition [20] consumed by the person [15] as applied towards a hypothetical person [15], John Doe during a period of one day, and of the step of compiling [60] a list [65] of the pre-treatment offending [23] and non-offending [22] sources of nutrition.

TABLE B

Identifying pre-treatment offending sources of nutrition [20] consumed by John Doe [15] during one day [73b] and compiling [60] a list [65] of the pre-treatment offending [23] and non-offending [22] sources of nutrition

| | Pre-treatment sources of nutrition [21] | Pre-treatment non-offending [22] and offending [23] sources of nutrition |
|---|---|---|
| Breakfast | | |
| 1. Drink | Mott’s 100% Apple Juice, which includes ascorbic acid | Offending |
| 2. Meat | Boar’s Head Blazing Buffalo chicken sausage by Brunckhorst, which includes vinegar | Offending |
| 3. Grains | Brownberry Natural Wheat Bread, which includes folic acid, yeast, vinegar, citric acid | Offending |
| | The Laughing Cow spreadable cheese wedges by Bel Brands USA, which includes cheddar, somisoft and swiss cheese, citric acid | Offending |
| Lunch | | |
| 1. Grains | Brew Pub Pizza Lotzza Motzza pepperoni pizza, which includes mozzarella cheese, folic acid, citric acid, yeast, romano cheese, citric acid, lactic acid | Offending |
| 2. Drink | Gold Peak Sweet Tea, which includes phosphoric acid | Offending |
| 3. Snack | Hot Pockets Sandwiches Four Cheese Pizza Garlic Buttery Crust, which includes mozzarella cheese, cheddar cheese, provolone cheese, parmesan cheese, citric acid, folic acid, yeast | Offending |
| 4. Dessert | Dannon Light & Fit Nonfat Strawberry Yogurt, which includes cultured milk, malic acid | Offending |
| Dinner | | |
| 1. Grains | Velveeta Original Shell Pasta and Creamy Cheese Sauce by Kraft Heinz Foods Company, which includes folic acid, lactic acid, sorbic acid, enzymes | Offending |
| 2. Meat | Gilbert’s fig and feta chicken sausage, which includes feta cheese, vinegar | Offending |
| 3. Salad | Organic Girl baby spring mix salad | Non-offending |
| | Kraft House Italian lite dressing, which includes vinegar, parmesan cheese, romano cheese, citric acid | Offending |
| 4. Drink | Wente Vineyards Chardonnay 2019 white wine | Offending |

Another step in the method of treating arthritis [10] is determining [70] a pre-treatment base level [75] of consumed offending sources of nutrition [23] of the person [15]. The outcome of this step [70] is obtaining a numerical value [75a] for the pre-treatment base level or load of offending sources of nutrition [75]. The pre-treatment base level [75] is a representation of the level, amount or severity of offending foods that the person [15] consumes prior to the treatment [23], and is represented using a numerical value [75a], a qualifier or a grouping. As an example, the pre-treatment consumption base level [75] takes the form of an index or a value [75a] that corresponds to the amount, strength or a combination of the amount and strength of the offending foods or sources of nutrition consumed by the person [23] during a pre-treatment consumption period of time [73b]. The pre-treatment consumption period of time [73b] is a period during which the identification of sources of nutrition for the person [20] occurs and it [73b] must be a finite period of time. For example, it is set to a day, week, two-weeks or month. The pre-treatment base level [75] allows for a comparison of how the aggregate consumption amount or load of offending sources of nutrition [23, 93] changes for the person from before treatment begins (pre-treatment) to after treatment begins (ongoing treatment or treatment).

One example of determining a pre-treatment base level [70] is to organize the pre-treatment offending sources of nutrition [23] for the person based on their degree of acidity or fermentation and designate [71] a strength factor [71a] for each offending source of nutrition [23] as shown in Table C below. In such case, each food, beverage or product from the list of pre-treatment sources of nutrition [65] for the person [15] is given a strength factor [71a] or, alternatively, is classified into tiers [71b] based on the degree of acidity or fermentation, from non-offending to extreme. When non-offending sources of nutrition [22] are included in the calculation [70] of the pre-treatment base level [75], they are given the strength factor value [71a] of zero. The strength factor [71a] is a binary indicator, 0 for non-offending and 1 for offending foods, or it is given a range of numerical values, for example, from 0 to 10 with 10 being very acidic or highly fermented. In the example where the strength factor [71a] is given values from 0 to 10 (Table C), the strength factor [71a] is given values of 0, 2, 3, 5 and 10, corresponding to non-offending, weak, medium, strong and extreme, respectively. The non-linear and increasing differences between consecutive values of the strength factor [71a] in that example reflects the non-linear, logarithmic nature of acidity and pH levels, as acidity strength increases exponentially with each one-point reduction in the pH value. The values given to the strength factor [71a] are ad hoc values as in the present example, or represent a measurement, for example, the measurement of the acidity of the food. The pre-treatment base level [75] is calculated as the average [71c] strength factor [71a] of all sources of nutrition consumed by the person [21] over a period of time [73b]. Table C provides an example of designating a strength factor [71] for some purposefully acidified foods and beverages [45].

TABLE C

Designating a strength factor [71] for some purposefully acidified foods, beverages and sources of nutrition [45]

| | Non-offending foods [22] | Offending foods [23] with some ingredients having acids added [45] | Offending foods [23] with most ingredients having acids added [45] | Offending foods [23] with all ingredients having acids added [45] | Offending foods [23] that are mainly acid [45] |
|---|---|---|---|---|---|
| | Non-offending | Weak | Medium | Strong | Extreme |
| | Strength factor [71a] | | | | |
| | 0 | 2 | 3 | 5 | 10 |
| Dairy | Cow milk, goat milk, sheep milk | | Flavored milk (strawberry) with acid added | | |
| Dairy cream | Cream, butter | Ice cream with toppings with acid added | Ice cream with all ingredients preserved with acids, butter with acids added | | |
| Alcohol | | | Flavored alcohol drinks (Corona hard seltzer), margarita made with margarita mix with acid added | | |
| Wheat | Regular wheat spaghetti | Pasta from flour with folic acid added | Macaroni or pasta with folic and other acid added | | |
| Meats | Fresh pork sausage, ribeye steak, pork and beef meat balls | Pork sausage with acid added, fresh meat with sauce with acid added | Meat marinated with acid | | |
| Drinks | Water, coffee, herbal tea | | Bottled coffee with acid added, apple juice with ascorbic acid added, orange juice with ascorbic acid added | Soft carbonated drink with acid added, cola with acid added, sports drink with acid added | |
| Fish | Fresh or frozen fish, fresh or frozen seafood | Fish with sorbic acid added | | | |
| Spreads | Fresh guacamole | Guacamole with ascorbic or other acid added | | | |
| Vegetables | Fresh cucumber, fresh vegetable | Canned vegetable with acid added, canned tomato with ascorbic or other acid added | | | |
| Bakery | Apple pie made without yeast, oatmeal cookie | Oatmeal cookie with filling with acid added, apple pie without yeast with filling with acid added | Cookies with filling where both have acid added, fruit bar with acid added, apple pie without yeast with acid added with filling with acid added | | |
| Dressing | Vegetable oil-based dressing, dairy cream-based dressing | | | | Vinegar-based dressing, vinegar-based sauce, vinegar, balsamic vinegar |
| Sauce | Dairy cream based sauce without fermented ingredients or acids | | | Ketchup made with vinegar | |

Table D provides an example of designating a strength factor [71] for some purposefully fermented foods, beverages and sources of nutrition [25].

TABLE D

Designating a strength factor [71] for some purposefully fermented foods, beverages and sources of nutrition [25]

| | Non-offending foods [22] | Offending foods [23] with some ingredients that are fermented [25] | Offending foods [23] with most ingredients that are fermented or with weak fermentation [25] | Offending foods [23] with all ingredients that are fermented or with strong fermentation [25] |
|---|---|---|---|---|
| | Non-offending | Weak | Medium | Strong |
| | Strength factor [71a] | | | |
| | 0 | 2 | 3 | 5 |
| Dairy | Cow milk, goat milk, sheep milk | Yogurt with jam or fruit topping | Young cheese, mozarella, cottage cheese, yogurt, kefir, buttermilk | Blue cheese, aged cheese |
| Dairy cream | Cream, butter | | Craime fraiche | Sour cream |
| Alcohol | | | Beer, cider | Whiskey, white wine, red wine |
| Wheat | Regular wheat spaghetti | | Bread, bun, pizza with young cheese | Pizza with blue or aged cheese |
| Meats | Fresh pork sausage, ribeye steak, pork and beef meat balls | | Salami, cured sausage | Aged sausage, cured and aged sausage |
| Drinks | Water, coffee, herbal tea | | Kvas, kombucha | |
| Spreads | Fresh guacamole | | Cheese-based spread | Blue-cheese-based spread |
| Dressing | Oil-based dressing, cream-based dressing | Oil-based dressings with cheese | | |
| Sauce | Cream based sauce without fermented ingredients or acids | | | Soy sauce |

Table E provides an example of designating a strength factor [71] for some foods, beverages and sources of nutrition that have been both purposefully fermented and purposefully acidified [55].

TABLE E

Designating a strength factor [71] for some foods, beverages and sources of nutrition that have been both purposefully fermented and purposefully acidified [55]

| | Non-offending foods [22] | Offending foods [23] with some ingredients that are fermented and with acids added [55] | Offending foods [23] with all ingredients that are fermented and with acids added [55] | Offending foods [23] with all ingredients that are acid and fermented [55] |
|---|---|---|---|---|
| | Non-offending | Medium | Strong | Extreme |
| | Strength factor [71a] | | | |
| | 0 | 3 | 5 | 10 |
| Dairy | Cow milk, goat milk, sheep milk | Yogurt with filling or topping with acid added, frozen yogurt with acid added | Cheese with ascorbic or citric acid added | |
| Wheat | Regular wheat spaghetti | | Bread with acid added, pizza with acid added with cheese with acid added | |
| Meats | Fresh pork sausage, ribeye steak, pork and beef meat balls | | Aged or cured sausage with acid added | |
| Drinks | Water, coffee, herbal tea | | Kvas with acid added, kombucha with acid added | |
| Spreads | Fresh guacamole | | Cheese spread with ascorbic, citric, or other acid added | |
| Vegetables | Fresh cucumber, fresh vegetable | | Pickled vegetable, pickled tomato, pickled cucumber | |
| Bakery | Apple pie made without yeast | | Apple pie with yeast with filling with acid added | |
| Dressing | Oil-based dressing, cream-based dressing | | Dressing that is fermented and preserved with acid | Vinegar-based dressing with blue cheese |
| Sauce | Cream based sauce without fermented ingredients or acids | | Sauce that is fermented and preserved with acid | Vinegar-based sauce that is fermented |

Table F provides an example of sources of nutrition for a person [21] and calculates the pre-treatment base level [75] as the average strength factor of sources of nutrition for the person [71c] over a pre-treatment consumption period [73b] of one day, where the strength factor [71a] is given values from 0 to 10.

TABLE F

Determining [70] pre-treatment base level [75] as the average strength factor [71c] for John Doe [15] during one day [73b]

| | Pre-treatment sources of nutrition [21] | Strength factor [71a] value [71b] |
|---|---|---|
| Breakfast | | |
| 1. Drink | Mott’s 100% Apple Juice, which includes ascorbic acid | 3 |
| 2. Meat | Boar’s Head Blazing Buffalo chicken sausage by Brunckhorst, which includes vinegar | 3 |
| 3. Grains | Brownberry Natural Wheat Bread, which includes folic acid, yeast, vinegar, citric acid | 5 |
| | The Laughing Cow spreadable cheese wedges by Bel Brands USA, which includes cheddar, somisoft and swiss cheese, citric acid | 5 |
| Lunch | | |
| 1. Grains | Brew Pub Pizza Lotzza Motzza pepperoni pizza, which includes mozzarella cheese, folic acid, citric acid, yeast, romano cheese, citric acid, lactic acid | 5 |
| 2. Drink | Gold Peak Sweet Tea, which includes phosphoric acid | 5 |
| 3. Snack | Hot Pockets Sandwiches Four Cheese Pizza Garlic Buttery Crust, which includes mozzarella cheese, cheddar cheese, provolone cheese, parmesan cheese, citric acid, folic acid, yeast | 5 |
| 4. Dessert | Dannon Light & Fit Nonfat Strawberry Yogurt, which includes cultured milk, malic acid | 3 |
| Dinner | | |
| 1. Grains | Velveeta Original Shell Pasta and Creamy Cheese Sauce by Kraft Heinz Foods Company, which includes folic acid, lactic acid, sorbic acid, enzymes | 5 |
| 2. Meat | Gilbert’s fig and feta chicken sausage, which includes feta cheese, vinegar | 5 |
| 3. Salad | Organic Girl baby spring mix salad | 0 |
| | Kraft House Italian lite dressing, which includes vinegar, parmesan cheese, romano cheese, citric acid | 10 |
| 4. Drink | Wente Vineyards Chardonnay 2019 white wine | 5 |

Pre-treatment base level [75] value [75a] 4.54

Another example of determining [70] the pre-treatment base level [75] is to apply consumption amounts (e.g., quantity, mass, weight, etc.) [72a] to the strength factors [71a] of the foods or sources of nutrition for the person [15] when calculating the base level [75]. In such implementation, the determination of the pre-treatment base level [70] consists of the following steps: a) designating a strength factor for each of the listed pre-treatment offending sources [71] (as described above), b) determining [72] pre-treatment average periodic (e.g., daily) consumption amount [72a] (e.g., quantity, mass, weight or an approximation thereof) for each listed pre-treatment offending source of nutrition for the person [65] in a pre-treatment consumption time period [73b] (e.g., daily, weekly, bi-weekly or monthly), c) determining [73] the pre-treatment weighted value [73a] for each listed pre-treatment offending source of nutrition [65] for the person [15] by multiplying the strength factor [71] numerical value [71a] by the average daily amount consumed [72a] in the pre-treatment consumption time period [73b], and d) adding [74] the weighted values [73a] of all offending sources of nutrition [65] for the person [15] in the pre-treatment consumption time period [73b]. The outcome of this step [70] is a numerical value [75a] of the pre-treatment base level [75] of all listed pre-treatment offending sources of nutrition for the person [65]. The determination [70] of a pre-treatment base level [75] using an average periodic consumption amount [72a] with a weighted strength factor [71a] for the person [15] is preferred to the determination [70] using the average strength factor of sources of nutrition for the person [71c].

An example of the determination [70] of a pre-treatment base level [75] using an average periodic consumption amount [72a] with a weighted strength factor [71a] for the person [15] is shown in Table G. Table G demonstrates the classification using a pre-treatment period [73b] of one day. Similar classifications are made for other finite periods, for example of one week, two weeks or a month.

TABLE G

Determining [70] pre-treatment base level [75] using a consumption [72a] quantity-weighted strength factor [71a] for John Doe [15] during one day [73b]

| | Pre-treatment sources of nutrition [21] | Pre-treatment consumption weight, grams [72a] | Strength factor [71a] value [71b] | Pre-treatment weighted value [73a] |
|---|---|---|---|---|
| Breakfast | | | | |
| 1. Drink | Mott’s 100% Apple Juice, which includes ascorbic acid | 240 | 3 | 720 |
| 2. Meat | Boar’s Head Blazing Buffalo chicken sausage by Brunckhorst, which includes vinegar | 84 | 3 | 252 |
| 3. Grains | Brownberry Natural Wheat Bread, which includes folic acid, yeast, vinegar, citric acid | 72 | 5 | 360 |
| | The Laughing Cow spreadable cheese wedges by Bel Brands USA, which includes cheddar, somisoft and swiss cheese, citric acid | 42 | 5 | 210 |
| Lunch | | | | |
| 1. Grains | Brew Pub Pizza Lotzza Motzza pepperoni pizza, which includes mozzarella cheese, folic acid, citric acid, yeast, romano cheese, citric acid, lactic acid | 132 | 5 | 660 |
| 2. Drink | Gold Peak Sweet Tea, which includes phosphoric acid | 547 | 5 | 2735 |
| 3. Snack | Hot Pockets Sandwiches Four Cheese Pizza Garlic Buttery Crust, which includes mozzarella cheese, cheddar cheese, provolone cheese, parmesan cheese, citric acid, folic acid, yeast | 120 | 5 | 600 |
| 4. Dessert | Dannon Light & Fit Nonfat Strawberry Yogurt, which includes cultured milk, malic acid | 150 | 3 | 450 |
| Dinner | | | | |
| 1. Grains | Velveeta Original Shell Pasta and Creamy Cheese Sauce by Kraft Heinz Foods Company, which includes folic acid, lactic acid, sorbic acid, enzymes | 112 | 5 | 560 |
| 2. Meat | Gilbert’s fig and feta chicken sausage, which includes feta cheese, vinegar | 142 | 5 | 710 |
| 3. Salad | Organic Girl baby spring mix salad | 142 | 0 | 0 |
| | Kraft House Italian lite dressing, which includes vinegar, parmesan cheese, romano cheese, citric acid | 31 | 10 | 310 |
| 4. Drink | Wente Vineyards Chardonnay 2019 white wine | 100 | 5 | 500 |
| | Total weight of food and beverage 1914 | | | |

Pre-treatment base level [75] value [75a] 8067

Another step in the method of treating arthritis [10] is altering [80] the pre-treatment offending sources of nutrition for the person [23] to treatment sources of nutrition [91]. The present method of treating arthritis [10] has as its primary method a reduction or elimination of the consumption of pre-treatment offending sources of nutrition [23]. To achieve that, one a) substitutes pre-treatment offending sources of nutrition [23] with alternative sources of nutrition [82], b) eliminates consumption of pre-treatment offending sources of nutrition [23] or c) reduces the amount consumed [72a] of the pre-treatment offending sources of nutrition [23]. With respect to the substitution of offending sources of nutrition [23] with alternative sources of nutrition [82], the alternative sources of nutrition [82] are treatment non-offending sources of nutrition [92] and treatment offending sources of nutrition [93]. The first preference is to substitute [84] pre-treatment offending sources of nutrition [23] with treatment non-offending sources of nutrition [92], that is, for alternative sources of nutrition [82] to be identified as non-offending [92]. The second preference is to substitute [86] pre-treatment offending sources of nutrition [23] with treatment offending sources of nutrition [93] that are classified with a lower or weaker strength factor [71a] when compared to the strength factor [71a] of the pre-treatment offending source of nutrition [23] that it substitutes. That is, if the alternative source of nutrition [82] is identified as offending [93], it is preferable that it has a lower or weaker strength factor [71a] relative to the strength factor [71a] of the pre-treatment offending source [23]. With respect to the quantity consumed [132] of the alternative source of nutrition [82], if the alternative source of nutrition [82] is identified as offending [93], the preference is to consume less than the amount consumed [72a] of the pre-treatment offending source [23] that is substitutes.

Another step in the method of treating arthritis [10] is identifying [88] the treatment offending sources of nutrition [93] consumed by the person [15]. Alternative sources of nutrition [82] that have been considered and selected for consumption are treatment sources of nutrition [91] for the person [15]. Treatment sources of nutrition [91] are identified as treatment non-offending sources of nutrition [92] and treatment offending sources of nutrition [93]. Treatment offending sources of nutrition [93] are identified similarly to the identification of pre-treatment offending sources of nutrition [20]. Treatment offending sources of nutrition [93] include treatment purposefully fermented foods or sources of nutrition [95], treatment purposefully acidified foods or sources of nutrition [96] and treatment purposefully fermented and acidified foods or sources of nutrition [97].

Another step in the method of treating arthritis [10] is compiling [100] a list [105] of listed treatment offending sources of nutrition [93] for the person [15], each of said listed treatment offending sources [93] being one of either the purposefully fermented sources [95], the purposefully acidified sources [96] and the both purposefully fermented and purposefully acidified sources [97]. The outcome of this step [100] is a list of listed treatment sources of nutrition that are identified as offending for the person [105]. The list [105] is used in the next step [110] of the method of treating arthritis [10].

Another step in the method of treating arthritis [10] is determining [110] a treatment base level [152] or treatment load of offending sources of nutrition [93] for the person [15]. The treatment base level [152] is a representation of the level, amount or severity of offending foods that the person consumes in the process of the treatment [93]. The algorithm for determining a treatment base level [110] should follow the algorithm for determining a pre-treatment base level [70]. Specifically, if the pre-treatment base level [75] is represented using a numerical value [75a], a qualifier or a grouping, the treatment base level [152] should be represented using a numerical value [154], a qualifier or a grouping. If the treatment base level [152] is represented using a numerical value [154], the outcome of this step us a treatment base level numerical value [154].

With respect to the first example provided for determining a pre-treatment base level [70], a corresponding way for determining a treatment base level [110] is to organize treatment offending sources of nutrition [93] based on their degree of acidity or fermentation and designate [120] a strength factor [122] for each of the listed treatment offending sources of nutrition [105] for the person [15]. Each food, beverage or product from the list of the treatment offending sources of nutrition for the person [105] is given a strength factor [122] (for example, from 0 to 10, with 10 being very acidic or fermented) or is classified into tiers [124] based on the degree of acidity or fermentation (from non-offending to extreme). The treatment base level [152] in this case equals an average strength factor [122] of listed sources of nutrition [105] for the person computed over a treatment consumption period of time [144] (a day, a week, a two-week period or a month). The outcome of this step [110] is a numerical value for the treatment base level [154] for the person [15].

With respect to the second example provided for determining a pre-treatment base level [70], a corresponding way for determining a treatment base level [110] is to calculate consumption amount-weighted [132] sum of strength factors [122] of listed treatment sources of nutrition for the person [105]. In such implementation, the determination of a treatment base level [110] consists of the following steps: a) designating a strength factor for each of said listed treatment offending sources [120], b) determining [130] treatment average daily consumption quantity, mass, weight or an approximation thereof [132] for each listed treatment offending sources of nutrition for the person [105] during the treatment consumption time period [144] (e.g., daily, weekly, bi-weekly or monthly), c) determining [140] the treatment weighted value [142] for each listed treatment offending source of nutrition [105] for the person by multiplying the strength factor [122] numerical value by average daily consumption quantity [132] of each offending source of nutrition in the treatment consumption time period [144], and d) adding [150] the weighted values [142] of all offending sources of nutrition for the person [105] in the treatment consumption time period [144] to obtain the numerical value [154] of the treatment base level [152].

The treatment steps of the method [79] (altering the consumption of offending foods or sources of nutrition [80], identifying the treatment offending foods or sources of nutrition consumed by the person [88], compiling a list of listed treatment offending sources of nutrition for the person [100] and determining the treatment base level [110]) must satisfy the condition that the treatment base level [152] does not exceed a lower tier fraction [158] of the pre-treatment base level [75] for the treatment consumption time period [144] (e.g., daily, weekly, bi-weekly or monthly). The lower tier fraction [158] is set depending on the implementation of the method [10]. An aggressive implementation [170] with the most immediate benefit for the person [15] is to eliminate consumption of offending foods or sources of nutrition [23] completely or to reduce the consumption as much as practically possible, and to do that quickly. In such implementation [170], the lower tier fraction [158] is set to be low from the start. However, it is possible that such implementation [170] is difficult and strenuous for the person [15] because of existing habits, preferences and tastes, which take effort and time to change. In part to account for these considerations, a tiered or stepwise implementation [180] reduces the lower tier fraction [158] gradually while monitoring the outcome. These implementations are discussed below.

In aggressive/hard-line strategy [170] implementation of the method [10], the lower tier fraction [158] is set low. For example, the lower tier fraction [158] is set to 5 percent. In such case, the treatment steps of the method [79] must satisfy the condition that the treatment base level [152] must not exceed 5% of the pre-treatment base level [75] for the treatment consumption time period [144]. The aggressive/hard-line strategy [170] is schematically demonstrated in FIG. 1 and an example is provided in Table H, in which the treatment base level [152] is reduced to less than 5 percent of the pre-treatment base level [75]. Specifically, in Table H, the treatment base level [152] is calculated as the consumption quantity [132] weighted sum of strength factors [122] of listed treatment offending sources of nutrition [105] over a period [144] of one day, and is reduced to 4.95% of the corresponding pre-treatment base level [75] of 8067 calculated in Table G.

TABLE H

Treatment base level [152] using a consumption [132] quantity-weighted strength factor [122] for John Doe [15] during one day [144] reduced to less than 5% of pre-treatment base level [75] under aggressive/hard-line strategy [170] implementation

| | Treatment sources of nutrition [91] | Treatment consumption weight, grams [132] | Strength factor [122] value [124] | Treatment weighted value [142] |
|---|---|---|---|---|
| Breakfast | | | | |
| 1. Drink | Bottled or tap water | 226 | 0 | 0 |
| 2. Meat | Usinger’s Uncured Beef Frankfurters by Usinger | 56 | 0 | 0 |
| 3. Grains | Angelic sprouted whole grain 7-grain bread, which includes yeast | 28 | 3 | 84 |
| | Sargento Sliced Medium Natural Cheddar Cheese, which includes cheese culture | 21 | 3 | 63 |
| Lunch | | | | |
| 1. Grains | La Campagne Chocolate Oatmeal Cookie | 104 | 0 | 0 |
| 2. Drink | Freshly brewed tea | 547 | 0 | 0 |
| 3. Snack | Angelic sprouted whole grain 7-grain bread, which includes yeast | 28 | 3 | 84 |
| | Boar’s Head Honey Smoked Turkey Breast | 113 | 0 | 0 |
| 4. Dessert | Haagen Dazs Vanilla Ice Cream | 236 | 0 | 0 |
| Dinner | | | | |
| 1. Grains | Delallo organic whole wheat spaghetti | 56 | 0 | 0 |
| | BelGioioso Parmesan Cheese, which includes cultured milk | 56 | 3 | 168 |
| 2. Meat | Baked chicken | 142 | 0 | 0 |
| 3. Salad | Organic Girl baby spring mix salad | 142 | 0 | 0 |
| | California Olive Ranch extra virgin arbequina olive oil | 30 | 0 | 0 |
| 4. Drink | Bottled or tap water | 226 | 0 | 0 |
| | Total weight of food and beverage 2011 | | | |

Treatment base level [152] value [154] 399
Fraction of pre-treatment base level 4.95%

Similarly, another example of the aggressive/hard-line strategy [170] implementation of the method [10] is provided in Table I in which the treatment base level [152] is calculated as the average strength factor [122] of treatment sources of nutrition [91]. This example corresponds to the pre-treatment base level [75] calculated as the average strength factor of sources of nutrition for the person [71c] provided in Table F. The treatment base level [152] calculated over a period [144] of one day is reduced to 4.41% of the corresponding pre-treatment base level [75] of 4.54 in Table F.

TABLE I

Treatment base level [152] using the average strength factor [122] for John Doe [15] during one day [144] reduced to less than 5% of pre-treatment base level [75] under aggressive/hard-line strategy [170] implementation

| | Treatment sources of nutrition [91] | Strength factor [122] value [124] |
|---|---|---|
| Breakfast | | |
| 1. Drink | Bottled or tap water | 0 |
| 2. Meat | Usinger’s Uncured Beef Frankfurters by Usinger | 0 |
| 3. Grains | Blue Diamond Nut Thins Rice Cracker Snacks Multi Seeds | 0 |
| | Sargento Sliced Medium Natural Cheddar Cheese, which includes cheese culture | 3 |
| Lunch | | |
| 1. Grains | La Campagne Chocolate Oatmeal Cookie | 0 |
| 2. Drink | Freshly brewed tea | 0 |
| 3. Snack | Blue Diamond Nut Thins Rice Cracker Snacks Multi Seeds | 0 |
| | Boar’s Head Honey Smoked Turkey Breast | 0 |
| 4. Dessert | Haagen Dazs Vanilla Ice Cream | 0 |
| Dinner | | |
| 1. Grains | Delallo organic whole wheat spaghetti | 0 |
| | California Olive Ranch extra virgin arbequina olive oil | 0 |
| 2. Meat | Baked chicken | 0 |
| 3. Salad | Organic Girl baby spring mix salad | 0 |
| | California Olive Ranch extra virgin arbequina olive oil | 0 |
| 4. Drink | Bottled or tap water | 0 |

Treatment base level [152] value [154] 0.20
Fraction of pre-treatment base level 4.41%

The next step under the aggressive or hard-line strategy [170] is maintaining [160] consumption of the treatment offending sources of nutrition [93] for the person at or below the treatment base level [152] in future consumption time periods. The person [15] follows this step for the rest of the person’s life or as long as is prescribed by the person’s physician.

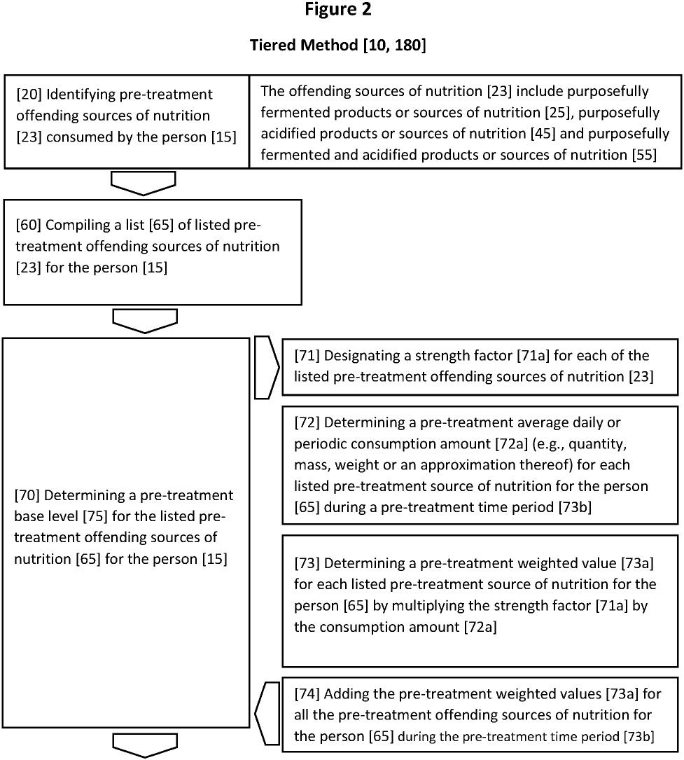

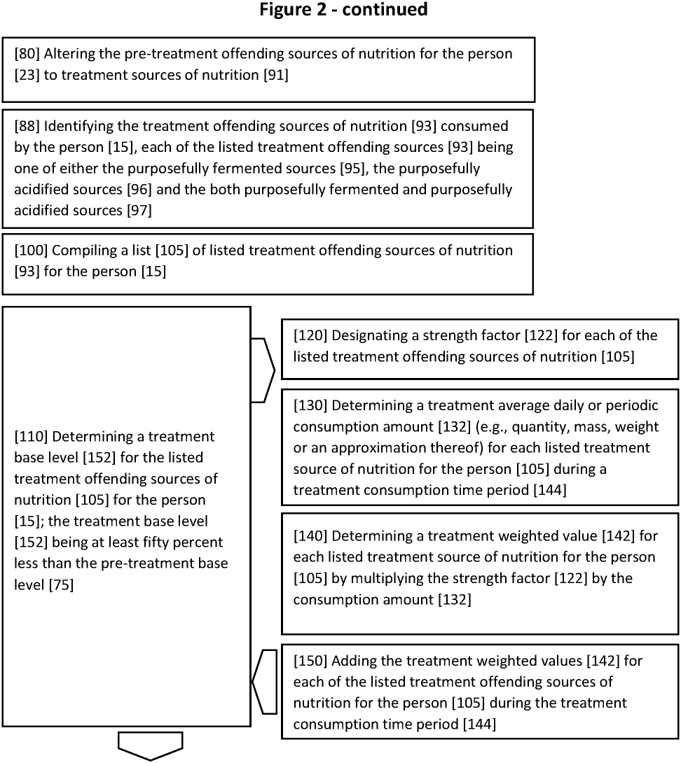

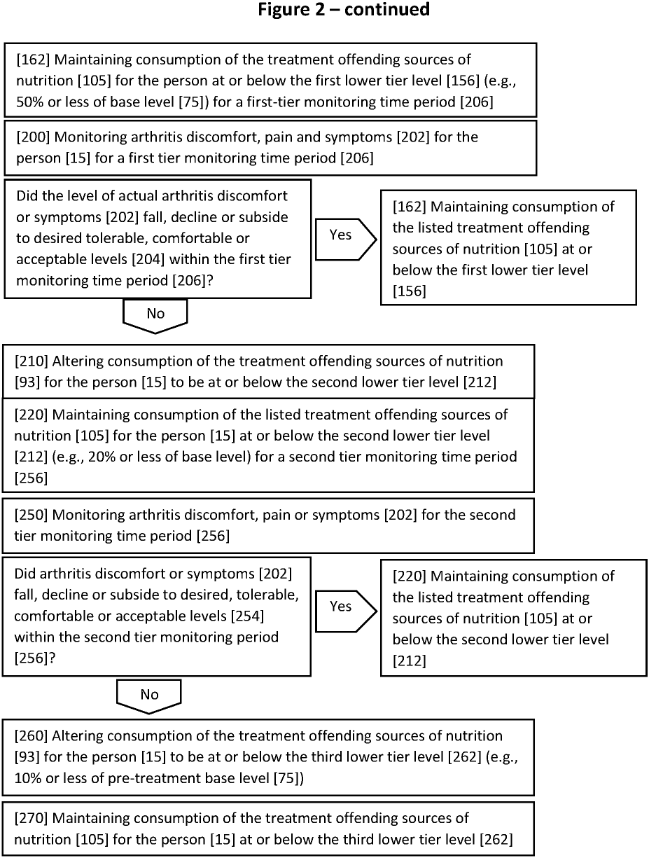

In tiered strategy [180] implementation of the method [10], the lower tier fraction [158] and the treatment base level [152] are lowered gradually, in a tiered manner. The tiered strategy [180] is schematically demonstrated in FIG. 2. In the first tier of the treatment steps of the method of treating arthritis [79], the lower tier fraction [158] is set to 50 percent, which imposes the condition that the treatment base level [152] must be 50% or less of the pre-treatment base level [75] for the treatment consumption time period [144]. The treatment base level [152] of 50% of the pre-treatment base level [75] is referred to as the first lower tier level [156]. An example of such implementation is provided in Table J, in which the treatment base level [152] is reduced to 34.44% (which is less than 50%) of the corresponding pre-treatment base level [75] of 8067 calculated in Table G.

TABLE J

Treatment base level [152] using a consumption [132] quantity-weighted strength factor [122] for John Doe [15] during one day [144] reduced to less than 50% (first lower tier fraction [158]) of pre-treatment base level [75] under tiered strategy [180] implementation

| | Treatment sources of nutrition [91] | Treatment consumption weight, grams [132] | Strength factor [122] value [124] | Treatment weighted value [142] |
|---|---|---|---|---|
| Breakfast | | | | |
| 1. Drink | Bottled or tap water | 226 | 0 | 0 |
| 2. Meat | Boar’s Head Blazing Buffalo chicken sausage by Brunckhorst, which includes vinegar | 84 | 3 | 252 |
| 3. Grains | Brownberry Natural Wheat Bread, which includes folic acid, yeast, vinegar, citric acid | 72 | 5 | 360 |
| | The Laughing Cow spreadable cheese wedges by Bel Brands USA, which includes cheddar, somisoft and swiss cheese, citric acid | 42 | 5 | 210 |
| Lunch | | | | |
| 1. Grains | Brew Pub Pizza Lotzza Motzza pepperoni pizza, which includes mozzarella cheese, folic acid, citric acid, yeast, romano cheese, citric acid, lactic acid | 132 | 5 | 660 |
| 2. Drink | Freshly brewed tea | 547 | 0 | 0 |
| 3. Snack | Hot Pockets Sandwiches Four Cheese Pizza Garlic Buttery Crust, which includes mozzarella cheese, cheddar cheese, provolone cheese, parmesan cheese, citric acid, folic acid, yeast | 120 | 5 | 600 |
| 4. Dessert | Haagen Dazs Vanilla Ice Cream | 236 | 0 | 0 |
| Dinner | | | | |
| 1. Grains | Barilla spaghetti, which includes folic acid | 56 | 2 | 112 |
| | BelGioioso Parmesan Cheese, which includes cultured milk | 28 | 3 | 84 |
| 2. Meat | Baked chicken | 142 | 0 | 0 |
| 3. Salad | Organic Girl baby spring mix salad | 142 | 0 | 0 |
| | California Olive Ranch extra virgin arbequina olive oil | 30 | 0 | 0 |
| 4. Drink | Wente Vineyards Chardonnay 2019 white wine | 100 | 5 | 500 |
| | Total weight of food and beverage 1957 | | | |

Treatment base level [152] value [154] 2778
Fraction of pre-treatment base level 34.44%

The next step in the tiered strategy [180] implementation of the method of treating arthritis [10] is composed of several individual steps [162, 200, 210, 220, 250, 260]. The maintaining step is maintaining [162] the consumption amount of treatment offending sources of nutrition [105] at or below the first lower tier level [156] (50% or less) for a first tier monitoring time period [206] (at least one week). The monitoring step is monitoring [200] a quantifiably determined level of arthritis discomfort, pain and symptoms [202] for the person [15] for the first tier monitoring time period [206] (at least one week). The first tier monitoring time period [206] during which the person [15] monitors arthritis discomfort, pain or symptoms [200] must be finite to provide a timeframe for the determination of the next step. In one possible implementation, the length or duration of the monitoring period of time [206] depends on the quantifiably determined level of arthritis discomfort, pain and symptoms of the person [202], with longer monitoring period of time [206] designated for more severe symptoms [202]. The person seeks for the level of arthritis discomfort or symptoms [202] to not exceed a desired quantifiable level [204] of tolerable, comfortable or acceptable pain and discomfort.

Determining the quantifiably determined level [202] of arthritis pain and discomfort of the person is based on one or more of the following factors, as is determining what constitutes a desired quantifiable level [204] of tolerable, comfortable or acceptable arthritis pain and discomfort. The determination of the person’s [15] actual level [202] of arthritis pain and discomfort and his or her desired level [204] of arthritis pain and discomfort is performed by the person either with or without the help and guidance of a physician. Quantifying and measuring these pain levels [202, 204] is based on an assessment that involves the frequency of arthritis pain and discomfort in the torso, neck, back, head, hands, arms, feet and legs, the duration of episodes of arthritis pain and discomfort in the torso, neck, back, head, hands, arms, feet and legs, the severity of arthritis pain and discomfort in the torso, neck, back, head, hands, arms, feet and legs, the limitations on the range of motion of the torso, neck, back, hands, arms, feet and legs that the pain and discomfort imposes, the number of sleep interruptions due to arthritis pain and discomfort, or the number and frequency of medications taken necessary to control or subdue arthritis pain and discomfort by the person [15]. For some persons, desired level [204] is no pain or discomfort whatsoever. For other persons, desired level [204] involves some tolerable pain or discomfort, depending on their activities, pain threshold and past experience. For still other persons, the level of desired, tolerable, comfortable or acceptable pain and discomfort changes over time. For example, someone initially in great pain first desires to reduce pain and is able to tolerate less severe pain. But as that person adjusts to less severe pain, that person wants to reduce the level of discomfort further, reducing the desired level of arthritis pain and discomfort [204].

If the actual or perceived levels of arthritis discomfort or symptoms [202] fall, decline or subside to desired, tolerable, comfortable or acceptable levels [204] within the first tier monitoring period [206], the next step in the tiered strategy [180] implementation of the method of treating arthritis [10] is to continue with maintaining consumption of treatment offending sources of nutrition at or below first lower tier level [162]. The person [15] follows this step for the rest of the person’s life or as long as is prescribed by the person’s physician.

If the actual or perceived levels of arthritis discomfort or symptoms [202] do not fall, decline or subside to desired, tolerable, comfortable or acceptable levels [204] within the first tier monitoring period [206], the next step is to proceed to the next lower tier level, which is the second lower tier level [212] (steps [210, 220, 250, 260]). The altering step is altering [210] consumption of the treatment offending sources of nutrition [93] for the person [15] to be at or below the second lower tier level [212]. Since the lower tier fraction [158] gets reduced in each subsequent tier, the second lower tier fraction [214] is less than the first lower tier fraction [158]. Consequently, the second lower tier level [212] is less than the first lower tier level [156]. For example, if the first lower tier fraction [158] is 50%, the second lower tier fraction [214] is 20%. In such case, the second lower tier level [212] is 20% of the pre-treatment base level [75]. An example of the implementation of the altering [210] step is provided in Table K. In Table K, the treatment base level [152] is reduced to 19.31% of the corresponding pre-treatment base level [75] of 8067 calculated in Table G.

TABLE K

Treatment base level [152] using a consumption [132] quantity-weighted strength factor [122] for John Doe [15] during one day [144] reduced to less than 20% (second lower tier fraction [214]) of pre-treatment base level [75] under tiered strategy [180] implementation

| | Treatment sources of nutrition [91] | Treatment consumption weight, grams [132] | Strength factor [122] value [124] | Treatment weighted value [142] |
|---|---|---|---|---|
| Breakfast | | | | |
| 1. Drink | Bottled or tap water | 226 | 0 | 0 |
| 2. Meat | Usinger’s Uncured Beef Frankfurters by Usinger | 56 | 0 | 0 |
| 3. Grains | Angelic sprouted whole grain 7-grain bread, which includes yeast | 56 | 3 | 168 |
| | Sargento Sliced Medium Natural Cheddar Cheese, which includes cheese culture | 42 | 3 | 126 |
| Lunch | | | | |
| 1. Grains | La Campagne Chocolate Oatmeal Cookie | 104 | 0 | 0 |
| 2. Drink | Freshly brewed tea | 547 | 0 | 0 |
| 3. Snack | Angelic sprouted whole grain 7-grain bread, which includes yeast | 56 | 3 | 168 |
| | Boar’s Head Honey Smoked Turkey Breast | 113 | 0 | 0 |
| 4. Dessert | Haagen Dazs Vanilla Ice Cream | 236 | 0 | 0 |
| Dinner | | | | |
| 1. Grains | Barilla spaghetti, which includes folic acid | 56 | 2 | 112 |
| | BelGioioso Parmesan Cheese, which includes cultured milk | 28 | 3 | 84 |
| 2. Meat | Baked chicken | 142 | 0 | 0 |
| 3. Salad | Organic Girl baby spring mix salad | 142 | 0 | 0 |
| | California Olive Ranch extra virgin arbequina olive oil | 30 | 0 | 0 |
| 4. Drink | Carlsberg beer | 300 | 3 | 900 |
| | Total weight of food and beverage 2134 | | | |

Treatment base level [152] value [154] 1558
Fraction of pre-treatment base level 19.31%

The next step is maintaining [220] consumption of the offending sources of nutrition [105] for the person [15] at or below the second lower tier level [212] (20% or less) for a second tier monitoring time period [256] (at least one week).

The next step is monitoring [250] the actual or perceived levels of arthritis discomfort, pain or symptoms [202] for the person [15] for a second tier monitoring time period [256]. The monitoring [250] step seeks to establish whether reduction of discomfort, pain or symptoms to desired levels [254] is achieved while maintaining consumption of listed treatment offending sources of nutrition [105] at or below the second lower tier level [212]. The second tier monitoring time period [256] must be finite to enable the determination of whether the desired, tolerable, comfortable or acceptable levels of pain and discomfort [254] has been achieved or whether to proceed to the next lower tier level.

If in monitoring [250] step, arthritis discomfort or symptoms fall to desired, tolerable, comfortable or acceptable levels [254] within the second tier monitoring time period [256], the next step in the tiered strategy [180] of the method of treating arthritis [10] is maintaining consumption of treatment offending sources of nutrition at or below second lower tier level [220]. The person [15] follows this step in the future for the rest of the person’s life or as long as is prescribed by the person’s physician.

If in monitoring [250] step arthritis discomfort or symptoms for the person [15] do not fall to desired, tolerable, comfortable or acceptable levels [254] within the second tier monitoring time period [256], the next step is altering [260] consumption of the treatment offending sources of nutrition [93] for the person [15] to be at or below the third lower tier level [262]. The third lower tier level [262] must be less than the second lower tier level [212]. For example, if the second lower tier fraction [214] is 20%, the third lower tier fraction [264] is 10% and, consequently, the third lower tier level [262] is 10% of the pre-treatment base level [75].

An example of the implementation of the altering [260] step is provided in Table L. In Table L, the treatment base level [152] is reduced to 9.20% of the corresponding pre-treatment base level [75] of 8067 calculated in Table G.

TABLE L

Treatment base level [152] using a consumption [132] quantity-weighted strength factor [122] for John Doe [15] during one day [144] reduced to less than 10% (third lower tier fraction [264]) of pre-treatment base level [75] under tiered strategy [180] implementation

| | Treatment sources of nutrition [91] | Treatment consumption weight, grams [132] | Strength factor [122] value [124] | Treatment weighted value [142] |
|---|---|---|---|---|
| Breakfast | | | | |
| 1. Drink | Bottled or tap water | 226 | 0 | 0 |
| 2. Meat | Usinger’s Uncured Beef Frankfurters by Usinger | 56 | 0 | 0 |
| 3. Grains | Angelic sprouted whole grain 7-grain bread, which includes yeast | 56 | 3 | 168 |
| | Sargento Sliced Medium Natural Cheddar Cheese, which includes cheese culture | 42 | 3 | 126 |

Lunch

| | | | | |
|---|---|---|---|---|
| 1. Grains | La Campagne Chocolate Oatmeal Cookie | 104 | 0 | 0 |
| 2. Drink | Freshly brewed tea | 547 | 0 | 0 |
| 3. Snack | Angelic sprouted whole grain 7-grain bread, which includes yeast | 56 | 3 | 168 |
| | Boar’s Head Honey Smoked Turkey Breast | 113 | 0 | 0 |
| 4. Dessert | Haagen Dazs Vanilla Ice Cream | 236 | 0 | 0 |
| Dinner | | | | |
| 1. Grains | Barilla spaghetti, which includes folic acid | 56 | 2 | 112 |
| | BelGioioso Parmesan Cheese, which includes cultured milk | 56 | 3 | 168 |
| 2. Meat | Baked chicken | 142 | 0 | 0 |
| 3. Salad | Organic Girl baby spring mix salad | 142 | 0 | 0 |
| | California Olive Ranch extra virgin arbequina olive oil | 30 | 0 | 0 |
| 4. Drink | Bottled or tap water | 226 | 0 | 0 |
| | Total weight of food and beverage 2088 | | | |

Treatment base level [152] value [154] 742
Fraction of pre-treatment base level 9.20%

The next step in the tiered strategy of the method is maintaining [270] consumption of the offending sources for the person [105] to at or below the third lower tier level [262] during future consumption time periods. The person [15] follows this step for the rest of the person’s life or as long as is prescribed by the person’s physician.

The implementation of the tiered strategy [180] of the method of treating arthritis [10] is not limited to three lower tier levels. In an alternative implementation, there are more than three lower tier levels. As an example of a five-tier implementation, lower tier levels one through five correspond to 50%, 35%, 20%, 10%, 5% of the pre-treatment base level [75], respectively. As an example of a six-tier implementation, lower tier levels one through six correspond to 50%, 35%, 20%, 10%, 5%, 1% of the pre-treatment base level [75], respectively.

An example of the 1% lower tier level is provided in Table M, in which the lower tier level equals 1% of the pre-treatment base level [75]. Specifically, the treatment base level [152] is reduced to 0.78% of the corresponding pre-treatment base level [75] of 8067 calculated in Table G.

TABLE M

Treatment base level [152] using a consumption [132] quantity-weighted strength factor [122] for John Doe [15] during one day [144] reduced to less than 1% of pre-treatment base level [75] under tiered strategy [180] implementation

| | Treatment sources of nutrition [91] | Treatment consumption weight, grams [132] | Strength factor [122] value [124] | Treatment weighted value [142] |
|---|---|---|---|---|
| Breakfast | | | | |
| 1. Drink | Bottled or tap water | 226 | 0 | 0 |
| 2. Meat | Usinger’s Uncured Beef Frankfurters by Usinger | 56 | 0 | 0 |
| 3. Grains | Blue Diamond Nut Thins Rice Cracker Snacks Multi Seeds | 30 | 0 | 0 |
| | Sargento Sliced Medium Natural Cheddar Cheese, which includes cheese culture | 21 | 3 | 63 |
| Lunch | | | | |
| 1. Grains | La Campagne Chocolate Oatmeal Cookie | 104 | 0 | 0 |
| 2. Drink | Freshly brewed tea | 547 | 0 | 0 |
| 3. Snack | Blue Diamond Nut Thins Rice Cracker Snacks Multi Seeds | 30 | 0 | 0 |
| | Boar’s Head Honey Smoked Turkey Breast | 113 | 0 | 0 |
| 4. Dessert | Haagen Dazs Vanilla Ice Cream | 236 | 0 | 0 |
| Dinner | | | | |
| 1. Grains | Delallo organic whole wheat spaghetti | 56 | 0 | 0 |
| | California Olive Ranch extra virgin arbequina olive oil | 30 | 0 | 0 |
| 2. Meat | Baked chicken | 142 | 0 | 0 |
| 3. Salad | Organic Girl baby spring mix salad | 142 | 0 | 0 |
| | California Olive Ranch extra virgin arbequina olive oil | 30 | 0 | 0 |
| 4. Drink | Bottled or tap water | 226 | 0 | 0 |
| | Total weight of food and beverage 1989 | | | |

Treatment base level [152] value [154] 63
Fraction of pre-treatment base level 0.78%

In an implementation of the method [10] in which the intention of the person [15] is to preventatively reduce the likelihood of future development of arthritis, osteoarthritis, rheumatoid arthritis, gout, low back pain and neck pain, the person [15] does not observe or feel pain, discomfort or symptoms. The monitoring [200] step is excluded and the person [15] proceeds to next lower tier level [212] [262] after monitoring time periods [206] [256].

The method for treating arthritis [10] is suited for implementation via electronic means, such as computers, laptops, smart phones, tables, smart watches, smart kitchen devices, electronic diaries, smart speakers, smart television sets, and software. These devices and software collect consumption data of the patient, identify pre-treatment offending sources of nutrition for the person [20], compile a list of listed pre-treatment offending sources of nutrition for the person [60], determine a pre-treatment base level of offending sources of nutrition for the person [70], alter the pre-treatment offending sources of nutrition for the person [80], compile a list of listed treatment offending sources of nutrition for the person [100], determine a treatment base level of offending sources for the person [110], monitor and help maintaining consumption of treatment offending sources at or below treatment base level for the person [160] or at or below lower tier levels, document and monitor the level of pain and discomfort of the person [200], and alter or help alter consumption of the treatment offending sources [210] in lower tier levels.

While the steps of determining [70, 110] pre-treatment and treatment base levels [75, 152] are preferred steps for practicing the dietary method for treating arthritis [10], it should be understood that the broad aspect of the dietary method can alter or avoid these steps. A more expedient form of the dietary method [10] includes the steps of identifying [20] the offending pre-treatment sources of nutrition [23], compiling [60] a list of listed offending pre-treatment sources of nutrition [23] with more offensive pre-treatment sources of nutrition being determined based on strength factors [71a] or tier levels [71b], altering [80] the pre-treatment offending sources of nutrition [23] to treatment sources of nutrition [91] and maintaining [160] consumption of the treatment sources of nutrition [91]. For example, in this more expedient method, the altering step [80] eliminates or reduces the consumption amount of a first set (e.g., 2 to 5 foods or beverages) of the more offensive pre-treatment offending sources of nutrition [23] to obtain the treatment sources of nutrition [91]. Should a monitoring step [202] determine that arthritis discomfort, pain and symptoms are not reduced to a tolerable level for this first lower tier level [156], then an additional altering step [210] further reduces or eliminates all consumption of the first set offending pre-treatment sources of nutrition [23] and can also further eliminate or reduce the consumption amount of a second set (e.g., an additional 2 to 5 foods or beverages) of the more offensive pre-treatment offending sources of nutrition [23] to obtain a second lower tier level [212] of the treatment sources of nutrition [91]. Further monitoring [250] and altering [260] steps are performed as needed until the person [15] observes a tolerable level of arthritis discomfort, pain and symptoms.

### Empirical Evidence Supports the Method of Treating Arthritis

Observations across populations provide evidence consistent with the subject method for treating arthritis. For instance, health problems are more prevalent in regions where people need to store and prevent the spoilage of foods. Across Europe, particularly historically, one can observe that populations of countries with access to fresh foods are in better health than those that rely on preserved foods. Nations that historically consume more fermented foods appear to have higher incidence of arthritis, gout, and other health problems.

Empirical evidence supports the method of treating arthritis by reducing the consumption of offending foods. Levels of different types of arthritis, skeletal, and joint diseases were evaluated across countries and related to food consumption. A populational approach is well suited to this type of examination because systemic conditions take time to develop, and, therefore, there is a certain lag between exposure to a causative agent and the resulting effect. Data was obtained on the prevalence of diseases and life expectancy from the Institute for Health Metrics and Evaluation’s Global Burden of Disease (GBD) Study 2017 (www.healthdata.org, University of Washington). Data on food consumption (so-called Food Balance Sheets) was obtained from the Food and Agriculture Organization (FAO) of the United Nations database FAOSTAT (www.foa.org). Data from the GBD are measured as the latest available data point between 1998 and 2000, while the FAO data are measured in 2014. These data bases span countries and various separate territories, but not all data are available across all countries. Data was used for 146 countries. The prevalence of diseases is measured in the number of cases per 100,000 of population, life expectancy is measured in years at birth, and food consumption is measured in food supply in kilograms per capita per year.

The year in which food consumption is measured differs from the year in which disease prevalence is measured. Data on a specific variable (for instance, the prevalence of arthritis) are captured around the same point in time, which provides appropriate relative ranking across countries. Although it is possible that food consumption changes over time (between the late 1990s and 2014), there is reason to suspect that due to culture, tradition, and habits, relative patterns of consumption across countries should exhibit a significant degree of inertia or stationarity. For instance, it is highly likely that the supply of beer and pig meat per capita has been higher in the United States of America than in the Kingdom of Saudi Arabia during both the late 1990s and the early 2010s. We derive additional assurance from the notion that if relative patterns in food consumption exhibited any change between the late 1990 and 2014, such change should bias out analysis against finding significant relations between the foods for which consumption changed and disease.

The examined variables of interest are the prevalence rates of diseases in a country’s population in cases per 100,000 of population. Several examined conditions covered by the GBD study are various forms of arthritis that have relatively high prevalence in the population, such as osteoarthritis, rheumatoid arthritis and gout. Low back pain and neck pain covered by the GBD study are also examined, which can be precursors of arthritis, an early form thereof, or an undiagnosed or weak form of ligament, joint or bone damage. The summary statistics for these variables are presented in Table 1. We report the means, the medians, the standard deviations, the 5th and the 95th percentiles of the distribution of the prevalence rate of each disease or condition.

TABLE 1

Summary statistics of the prevalence of arthritis

| in cases per 100,000 population | Osteoarthritis | Rheumatoid arthritis | Prevalence of Gout | Low back pain | Neck Pain |
|---|---|---|---|---|---|
| | (1) | (2) | (3) | (4) | (5) |
| Mean | 2,816 | 215 | 419 | 8,407 | 3,054 |
| Median | 2,207 | 174 | 284 | 6,398 | 2,376 |
| Standard deviation | 1,736 | 134 | 289 | 4,267 | 1,552 |
| 5th percentile | 944 | 66 | 166 | 4,263 | 1,417 |
| 95th percentile | 6,059 | 479 | 967 | 15,885 | 6,458 |

Table 1 demonstrates two main points. First, arthritis-related conditions and diseases are prevalent. The average prevalence rate of osteoarthritis is 2,816 or 2.82%, of rheumatoid arthritis is 215 or 0.21%, and of gout is 419 or 0.42%. The prevalence of low back pain is even higher, with the average rate of 8,407 or 8.40% of the population being affected. Second, the prevalence rates vary substantially from country-to-country. Countries in the 95th percentile have about five times the prevalence rate of the countries in the 5th percentile. For example, for osteoarthritis, the 5th percentile of prevalence is 944 or 0.94% ( e.g., Kenya), and the 95th percentile of prevalence is 6,069 or 6.07% (e.g., Belgium). For low back pain, the 5th percentile of prevalence is 4,263 or 4.26% (e.g., Ghana), and the 95th is 15,885 or 15.88% (e.g., Romania). For neck pain, the 5th percentile is 1,417 or 1.42% (e.g., Mozambique), and the 95th percentile is 6,458 or 6.46% (e.g., Switzerland). The variation in disease prevalence from country-to-country reveals that various factors may contribute to the differences in the rates of prevalence, and these variations are analyzed using statistical methods.

As regards explanatory variables, or the variables that represent factors that may correlate with or determine the diseases, data is used form of the grouping of foods and categories of foods by FAO. Were data is not directly available for some foods, approximations or proxies are used. Beer and wine per capita per year are examined because they both represent foods that are both acidic and fermented foods, but to varying degrees. For regression analysis, Additional variables that serve as “control” variables are used that correlate with disease prevalence, such as life expectancy at birth, and supply quantity of other alcoholic beverages, vegetables, cereal grains, meat, sugar, and fruit. Variables that measure disease prevalence and variables that measure food consumption are calculated on a per capita basis, which enables comparisons of countries of various sizes and populations, and allows the inclusion of observations from various countries in the same analysis and regression model.

### Pair-wise Summary Statistics Comparisons

Before examining the data using regression analysis, pair-wise comparison is performed for diseases and food consumption between some countries in Europe. Europe provides a good ground for such comparisons because a relatively compact and uniform territory is broken up into many countries that differ in their food traditions and preferences. For example, countries that are similar and close geographically but differ in the amounts of food consumed are Greece and Croatia, and Denmark and Estonia. The statistics of the supply of wine and beer and the prevalence of diseases for these countries are reported in Table 2.

TABLE 2

Summary statistics of the prevalence of arthritis and of the supply of fermented beverages

| Country | Supply of in kg/capita/yr | | Prevalence of in cases per 100,000 population | | | | |
|---|---|---|---|---|---|---|---|
| | Wine | Beer | Osteo arthritis | Rheumatoid arthritis | Gout | Low back pain | Neck Pain |
| Greece | 18.27 | 36.11 | 6,209 | 452 | 967 | 14,734 | 6,278 |
| Croatia | 13.38 | 80.99 | 4,597 | 307 | 560 | 15,673 | 4,095 |
| Denmark | 29.12 | 61.68 | 5,974 | 491 | 910 | 15,833 | 7,118 |
| Estonia | 14.53 | 100.38 | 4,784 | 366 | 589 | 13,311 | 4,042 |

When compared to Croatia, Greece has higher wine consumption per capita (18.27 kilograms versus 13.38), but much lower beer consumption (36.11 kilograms versus 80.99). Despite significantly lower beer consumption and sharing a favorable Mediterranean location, Greece has higher prevalence of arthritis. The rate of rheumatoid arthritis is 452 per 100,000 population in Greece versus 306 in Croatia, the rate of osteoarthritis is 6,209 in Greece versus 4,597 in Croatia, gout is 967 versus 560, low back pain is 14,734 and 15,673, and neck pain is 6,278 and 4,095, respectively. The rates for all arthritis-related conditions are worse in Greece, with the exception of only low back pain, which has comparable prevalence in both countries.

Denmark and Estonia share a similar pattern. The Danes favor wine much more than the Estonians, with per capita consumption of 29.12 in Denmark versus 14.53 in Estonia. Both countries drink beer, 61.68 versus 100.38, respectively. In Denmark, however, the rates of all arthritis-related conditions are higher than those in Estonia: rheumatoid arthritis is 491 versus 366, osteoarthritis is 5,974 versus 4,784, gout is 910 versus 589, low back pain is 15,833 and 13,311, and neck pain is 7,118 and 4,042 in Denmark and Estonia, respectively. In both comparisons, the rates of arthritis-related conditions are higher in the country with higher wine consumption, not the one with the higher beer consumption. In both comparisons, the countries are comparable in their geographic locations, educational level of population, hygiene and access to basic medical care and clean water. But they differ in their preference for drinks, and, possibly, this in part determines the differences in the rates of some diseases that their populations are affected by. This evidence does not prove causality, but it serves as a first step in revealing and documenting a relation between arthritis prevalence and consumption of specific foods. To provide a more formal comparison across many countries while simultaneously controlling for consumption of other foods, we turn to a methodology that allows us to do so, namely, regression analysis.

### Regression Analysis - Baseline Model

Regression analysis examines correlations between variables while simultaneously accounting for the effect of other variables, and shows how the level of disease across countries correlates with the consumption of certain foods. An Ordinary Least Squares (OLS) regression approach is superior to correlation coefficients or summary statistics because it controls for other factors and variables that may correlate with disease prevalence. Statistical inference in the analysis is calculated using standard errors assuming normal distribution of the error term. Cross-sectional data is utilized without the time-series component, which alleviates concerns about autocorrelation. The data are also measured on a per capita level, alleviating the effect of country size and concerns about heteroskedasticity.

To begin, the prevalence of arthritis-related conditions utilizing only the supply of wine and beer as explanatory variables is examined. Table 3 shows coefficient estimates from the model, along with the statistical significance of the estimates denoted by asterisks, and standard errors of the estimates reported in parentheses below the estimates. With respect to statistical significance, three asterisks (“***”) correspond to a p-value of 0.01 or less, and denote that the coefficient is significantly different from the null hypothesis of zero at the 1% level, two asterisks (“**”) correspond to a p-value of 0.05 or less, and denote statistical significance at the 5% level, and one asterisk (“*”) corresponds to a p-value of 0.1 or less, and denotes statistical significance at the 10% level. Three asterisks denote the highest level of significance conventionally adopted in research, while one asterisk denotes a lower level, although still statistically significant at conventional level as adopted in research. Each observation in the analysis uses data for individual country, and the analysis uses data from 145 or 146 countries.

TABLE 3

Regression analysis of the prevalence of arthritis
Dependent Variable: Prevalence of

| Model | Osteoarthritis | Rheumatoid arthritis | Gout | Low back pain | Neck pain |
|---|---|---|---|---|---|
| | (1) | (2) | (3) | (4) | (5) |
| Wine | 100.371^{∗∗∗} (10.58) | 7.235^{∗∗∗} (0.81) | 17.658^{∗∗∗} (1.86) | 219.462^{∗∗∗} (26.36) | 100.942^{∗∗∗} (9.75) |
| Beer | 18.406^{∗∗∗} (3.44) | 1.648^{∗∗∗} (0.26) | 2.453^{∗∗∗} (0.61) | 55.483^{∗∗∗} (8.57) | 11.035^{∗∗∗} (3.17) |
| Constant | 1,574.145.00^{∗∗∗} (129.40) | 114.968^{∗∗∗} (9.87) | 226.854^{∗∗∗} (22.76) | 5,211.164.00^{∗∗∗} (322.35) | 2,025.338.00^{∗∗∗} (119.24) |
| Observations | 146 | 146 | 146 | 146 | 146 |
| Adjusted R-squared | 62.05% | 63.03% | 58.23% | 61.49% | 59.88% |

The first model (Model 1) in Table 3 pertains to the prevalence of osteoarthritis. The coefficient estimate of the supply of wine is 100.371 and the coefficient estimate of the supply of beer is 18.406. Both are positive and statistically significant with a p-value of less than 0.01. This rejects null hypothesis that the prevalence of osteoarthritis is unrelated to the supply of wine or the supply of beer. The results support the subject method that higher average per capita supply of wine and beer in a country is positively associated with the prevalence of osteoarthritis, and suggesting that higher consumption of wine or beer aggravates or even causes the condition. The effect of the consumption of wine is much stronger than the effect of the consumption of beer, as the coefficient of wine (100.371) is about five and a half times (=100.371/18.406) greater than the coefficient of beer (18.406).

The other four models in Table 3 pertain to other forms of arthritis and conditions related to arthritis. Specifically, Models 2-5 pertain to the prevalence of rheumatoid arthritis, gout, low back pain, and neck pain. The coefficients of the supply of wine and beer are generally in line with that of osteoarthritis. All of the coefficients of wine and beer are positive and statistically significant with a p-value of less than 0.01. The coefficients of wine are also several times greater than the coefficients of beer. The ratio of the magnitude of the coefficients of wine to the coefficients of beer are 4.39 (7.235/1.648) for rheumatoid arthritis, 7.20 (17.658/2.453) for gout, 3.95 (219.462/55.483) for low back pain, and 9.15 (100.942/11.035) for neck pain. Depending on the form of arthritis, the correlation with the supply of wine is four to nine times stronger than the correlation with the supply of beer.

One or more of the characteristics of these beverages show a positive correlation with arthritis. One obvious characteristic of wine and beer is alcohol content. Yet, alcohol content does fully explain the correlation of wine and beer to arthritis. If it did, the difference in alcohol content would match the difference in the coefficient estimates. That is, because the coefficients of wine are four to nine times greater than the coefficients of beer, a kilogram of wine should contain four to nine times the amount of alcohol than a kilogram of beer. However, the effect of wine as compared to the effect of beer is much greater than the amount of alcohol indicates. The typical alcohol content (alcohol by volume) for wine is about 12.5% to 14.5%, and for beer about 4% to 6%, with the average being about 13.5% and 5%, respectively. Wine and beer have comparable mass densities, or similar weight per volume, their alcohol by volume is directly comparable. If the full effect were due to alcohol, we expect the effect of wine to be about 2.7 (13.5%/5%) times greater than that of beer. Yet, the ratio of coefficient estimates varies between 4.39 (=7.235/1.648) for rheumatoid arthritis and 9.15 (100.942/11.035) for neck pain, indicating that the difference between the effects of wine and beer are much greater than the alcohol content implies. Therefore, alcohol content alone does not fully explain the difference in the correlation of wine and beer with the prevalence of arthritis.

Wine and beer also differ in fermentation and acidity. Compared to beer, wine is typically subject to longer fermentation and has higher acidity. The acidity of typical wine ranges from 3.5 to 4, whereas that of beer ranges around 4 to 5. Since the scale is logarithmic, the acidity of wine is around 3 to 10 times higher than that of beer. The differences in acidity between wine and beer are in line with the differences in the coefficient estimates of wine and beer in arthritis regressions. This is consistent with fermentation and acidity explaining a higher correlation of wine with the prevalence of arthritis. First, fermentation and acidity characteristics also explain the positive correlations between wine and arthritis, as well as beer and arthritis. Second, the results of our empirical analysis are consistent with the subject method in that foods which underwent fermentation or which have higher acidity are positively correlated the prevalence of arthritis. The following empirical results are consistent with the method for treating arthritis in that wine and beer are positively correlated with the prevalence of arthritis, and the difference in the degree of correlation is comparable to the difference in their acidity and fermentation, but is not comparable to the difference in their alcohol content. Contrary to the histamine intolerance literature discussed above, this evidence is consistent with the subject method in that the adverse effect of wine is more adverse than that of beer and this trend exists in a much wider population. The evidence is consistent with the subject method in that the adverse effects of wine, beer, and potentially other fermented foods extend into the wider population, particularly those affected by or susceptible to arthritis.

Table 3 provides two other types of estimates: a) coefficients of the constant, and b) adjusted R-squared. Both provide information about the magnitudes of the relations between dependent variables and explanatory variables. The coefficient of the constant (1,574.145.00 in model 1) estimates a hypothetical point of the intersection of the fitted function of the model with the axis of the dependent variable. It provides an estimate of the average prevalence rate of arthritis under the scenario in which consumption of wine and beer is zero. Under this scenario, the average prevalence rate of osteoarthritis would be around 1,574. That is about 44% lower than the actual average prevalence rate of 2,816, reported in Table 1. In an average country in which no wine or beer are consumed, the prevalence rate of osteoarthritis should be about half of the rate of a country with average consumption of wine and beer. In other words, if the consumption of wine and beer were to decline to zero, the prevalence rate of osteoarthritis would drop by almost half. Using similar analysis, the coefficients of the constant are 114.968 for rheumatoid arthritis and 226.854 for gout, predicting that the average prevalence for both would fall by about 46%, from 215 to about 115 and from 419 to about 227, respectively. For low back pain and neck pain the predicted reductions in the prevalence is lower, but still material at 38% (from 8,407 to 5,211.164.00) and 33% (from 3,054 to 2,025.338.00), respectively.

Another way to assess the absolute magnitude of the effects is to estimate the change in the dependent variable that results from a one-standard-deviation change in the explanatory variable. Focusing on the prevalence of osteoarthritis and its correlation with the supply of wine, the coefficient estimate is 100.371 in model 1. Given that the mean of the supply of wine is 6.3 and the standard deviation is 9.8 kilograms per capital per year, one standard deviation change in the supply of wine changes the prevalence of osteoarthritis by 983.64 (9.8 × 100.371), which is about 35.0% (983.64/2,816) of the average prevalence of osteoarthritis. Similarly, for rheumatoid arthritis and gout, a one standard deviation change in the supply of wine changes disease prevalence by 70.90 (9.8 × 7.235, the coefficient obtained from model 2) and 173.05 (9.8 × 17.658, the coefficient obtained from model 3), which are 33.0% (70.90/215) and 41.3% (173.05/419) of the average prevalence rate of rheumatoid arthritis and gout, respectively. For low back pain and neck pain, one-standard-deviation change in the supply of wine results in the change of prevalence of 25.6% (9.8×219.462/8,407) and 32.4% (9.8×100.942/3,054), respectively.

The other type of estimates reported in Table 3 is the estimate of the adjusted R-squared, which ranges from 58.23% in model 3 to 63.03% in model 2. The R-squared measures the proportion of the variation in the dependent variable that is explained by the independent variables. More than half of the variation in the prevalence rate of arthritis is explained by the consumption of wine and beer, and the constant. To estimate the contribution of each individual variable to the R-squared, each variable is excluded and a drop in the adjusted R-squared is observed. For the consumption of wine, it is around 16%, while for the consumption of beer, it is around 8% in model 1. This provides additional evidence that the relations between the prevalence rates of arthritis and the consumption of wine and beer across countries is materially significant, and consistent with the subject method for treating arthritis by reducing the consumption of wine and beer.

Overall, the above results are consistent with the subject method that a reduction in the consumption of wine and beer will provide material benefits to those already affected by arthritis, and potentially prevent arthritis in those who are otherwise likely to be affected by it. Furthermore, reductions in the consumption of other fermented and acidic foods likely also result in lower prevalence of arthritis. The above results for low back pain and neck pain are similar to that for analysis of osteoarthritis, rheumatoid arthritis and gout. This indicates that the relations and possible effects observed in the population with arthritis extend into the more general population with skeletal or joint pains.

### Regression Analysis - Model With Additional Control Variables

To provide additional tests on whether the results are due to the effect of alcohol, and to control for additional foods and factors that may affect disease prevalence, additional factors are included into the model. The supply of other alcoholic beverages, fruit, vegetables, cereal grains, meat, and sugar are included. In addition, to control for the general health and quality of life in the country, the average life expectancy at birth in the country in 1999 is included. The results of the regressions are reported in Table 4.

TABLE 4

Regression analysis of the prevalence of arthritis
Dependent Variable: Prevalence of

| Model | Osteoarthritis | Rheumatoid arthritis | Gout | Low back pain | Neck pain |
|---|---|---|---|---|---|
| | (6) | (7) | (8) | (9) | (10) |
| Wine | 69.432^{∗∗∗} (9.52) | 5.269^{∗∗∗} (0.77) | 13.055^{∗∗∗} (1.86) | 164.934^{∗∗∗} (26.00) | 80.911^{∗∗∗} (9.38) |
| Beer | 7.302^{∗∗} (3.50) | 0.840^{∗∗∗} (0.28) | 0.983 (0.68) | 41.530^{∗∗∗} (9.56) | 7.170^{∗∗} (3.45) |
| Alcoholic beverages | 29.414 (21.88) | 2.266 (1.77) | -4.79 (4.27) | 26.937 (59.74) | -4.837 (21.56) |
| Vegetables | 1.729 (1.36) | -0.006 (0.11) | 0.460^{∗} (0.27) | 7.726^{∗∗} (3.71) | 3.992^{∗∗∗} (1.34) |
| Cereal | -0.192 (1.74) | 0.097 (0.14) | -0.314 (0.34) | 7.981^{∗} (4.75) | 1.291 (1.71) |
| Meat | 8.065^{∗} (4.50) | 0.584 (0.36) | 2.862^{∗∗∗} (0.88) | 11.111 (12.28) | 0.598 (4.43) |
| Sugar | -4.001 (7.38) | 1.173^{∗} (0.60) | -0.73 (1.44) | -35.902^{∗} (20.14) | -6.308 (7.27) |
| Life expectancy | 71.804^{∗∗∗} (13.48) | 3.161 ^{∗∗∗} (1.09) | 6.278^{∗∗} (2.63) | 132.187^{∗∗∗} (36.80) | 53.136^{∗∗∗} (13.28) |
| Fruit total | -3.823^{∗∗} (1.52) | 0.069 (0.12) | -0.889^{∗∗∗} (0.30) | -9.517^{∗∗} (4.16) | -2.681^{∗} (1.50) |
| Constant | -2,919.347.00^{∗∗∗} (737.62) | -140.081^{∗∗} (59.68) | -158.026 (143.80) | -3,360.563.00^{∗} (2013.87) | -1,431.704.00^{∗} (726.80) |
| Observations | 145 | 145 | 145 | 145 | 145 |
| Adjusted R-squared | 75.86% | 73.49% | 67.35% | 70.57% | 70.84% |

There are several notable findings. First, even after controlling for a list of additional factors, the differences between the coefficients of wine and beer remain high. The ratio of the coefficients equals 9.51 (69.432/7.302) for osteoarthritis, 6.27 (5.269/0.840) for rheumatoid arthritis, 13.28 (13.055/0.983) for gout, 3.97(164.934/41.530) for low back pain, 11.28 (80.911/7.170) for neck pain. The average ratio is 8.86, which indicates that the effect of wine is much more adverse as compared to the effect of beer. Second, the supply of other alcoholic beverages is not significantly related to the prevalence of arthritis. Not one of the five coefficients (coefficient of 29.414 in model 6, 2.266 in model 7, -4.790 is model 8, 26.937 in model 9 and -4.837 in model 10) is statistically significant at conventional levels. These results reinforce the notion that alcohol content alone is unlikely to fully explain the correlation between wine, beer, and arthritis. The result supports the subject method and that fermentation and acidity determine the correlation of wine and beer with arthritis.

Finally, there is evidence that fruit are generally associated with lower disease prevalence, with negative and statistically significant coefficients in four out of five models (-3.823 in model 6, -0.889 in model 8, -9.517 in model 9, and -2.681 in model 10). The acidity balance literature argues that although most fruit are acidic, citrus fruit, which include lemons, limes, oranges, grapefruits, mandarins, and tangerines, among others, have a strong alkaline effect. The acidity balance literature particularly stresses the beneficial effect of lemons. In order to examine this argument directly, we partition the supply of fruit into citrus fruit and other fruit, estimate the model with these two variables in place of one aggregate fruit variable, and report results in Table 5.

TABLE 5

Regression analysis of the prevalence of arthritis
Dependent Variable: Prevalence of

| Model | Osteoarthritis | Rheumatoid arthritis | Gout | Low back pain | Neck pain |
|---|---|---|---|---|---|
| | (11) | (12) | (13) | (14) | (15) |
| Wine | 69.067^{∗∗∗} (9.53) | 5.225^{∗∗∗} (0.77) | 13.006^{∗∗∗} (1.86) | 164.998^{∗∗∗} (26.12) | 80.574^{∗∗∗} (9.39) |
| Beer | 7.857^{∗∗} (3.54) | 0.907^{∗∗∗} (0.29) | 1.057 (0.69) | 41.430^{∗∗∗} (9.71) | 7.683^{∗∗} (3.49) |
| Alcoholic beverages | 25.728 (22.16) | 1.82 (1.78) | -5.286 (4.33) | 27.596 (60.75) | -8.246 (21.85) |
| Vegetables | 1.884 (1.37) | 0.013 (0.11) | 0.480* (0.27) | 7.698** (3.75) | 4.135^{∗∗∗} (1.35) |
| Cereal | -0.481 (1.76) | 0.062 (0.14) | -0.353 (0.34) | 8.032^{∗} (4.83) | 1.024 (1.74) |
| Meat | 8.027^{∗} (4.50) | 0.579 (0.36) | 2.857^{∗∗∗} (0.88) | 11.119 (12.32) | 0.562 (4.43) |
| Sugar | -5.321 (7.48) | 1.013^{∗} (0.60) | -0.908 (1.46) | -35.665^{∗} (20.51) | -7.528 (7.38) |
| Life expectancy | 71.269^{∗∗∗} (13.48) | 3.096^{∗∗∗} (1.09) | 6.206^{∗∗} (2.63) | 132.284^{∗∗∗} (36.96) | 52.642^{∗∗∗} (13.29) |
| Fruit excluding citrus | -4.899^{∗∗∗} (1.84) | -0.061 (0.15) | -1.034^{∗∗∗} (0.36) | -9.325^{∗} (5.05) | -3.675^{∗∗} (1.82) |
| Citrus fruit | -1.017 (3.11) | 0.409 (0.25) | -0.511 (0.61) | -10.019 (8.52) | -0.086 (3.07) |
| Constant | -2,813.665.00^{∗∗∗} (744.45) | -127.296^{∗∗} (59.93) | -143.796 (145.43) | -3,379.477.00 (2040.59) | -1,333.987.00^{∗} (733.88) |
| Observations | 145 | 145 | 145 | 145 | 145 |
| Adjusted R-squared | 75.87% | 73.76% | 67.23% | 70.35% | 70.83% |

The results reveal that four of five coefficients of citrus fruit are negative (-1.017 in model 11, -0.511 in model 13, -10.019 in model 14, -0.086 in model 15) and only one positive (0.409 in model 12). Importantly, none of these coefficients is statistically significant. In contrast, the coefficients of fruit that are not citrus are all negative (-4.899 in model 11, -0.061 in model 12, -1.034 in model 13, -9.325 in model 14, -3.675 in model 15) and four out of five are statistically significant. It appears that higher supply of fruit that are not citrus is associated with a reduction in arthritis prevalence. Notably, higher supply of citrus fruit is not associated with a reduction in arthritis prevalence. This is inconsistent with the arguments of the acidity balance literature about the effect of citrus fruit. Furthermore, the evidence further uncovers limitations of the general notion underlying the acidity balance literature, as the absolute magnitude of the negative coefficients of non-citrus fruit is much smaller, less than one-tenth, of the absolute magnitude of the positive coefficients of wine, for example. This is consistent with the notion that if the correlations represent causal relations between foods and arthritis, the reduction in the consumption of fermented and acidic foods reduce the likelihood of developing arthritis, alleviate existing arthritis, and alleviate future disease progression.

Overall, the results of the empirical analysis indicate that higher average per capita supply of wine and beer in a country is associated with higher prevalence of various forms of arthritis, low back pain, and neck pain. This is consistent with the subject method and that higher consumption of wine and beer aggravates or even causes the condition. Additionally, the effect of the consumption of wine is much stronger than the effect of the consumption of beer. The differences in the magnitude of the effect between wine and beer align with the differences in fermentation and acidity of wine and beer. This is consistent with the subject method and that the correlations between wine and arthritis, and beer and arthritis are determined by fermentation and acidity. Under the notion that the correlations reflect a causal relation between wine, beer and arthritis, a reduction in the consumption of wine and beer leads to a reduction of the prevalence of arthritis, low back pain, and neck pain. In other words, the evidence implies that a reduction in the consumption of wine and beer alleviates symptoms of existing arthritis and skeletal and joint pains and damage, reduces further disease progression, and reduces the development of arthritis and skeletal and joint pains and damage in those yet unaffected. The evidence further implies that a reduction in the consumption of other fermented or acidic foods has similar therapeutic and preventative effects. That is, the evidence implies that the reduction in the consumption of acidic and fermented foods provides a benefit to those diagnosed with an arthritis condition and reduces arthritis in the future by those who have not developed the condition or who have not been diagnosed.

While the invention has been described with reference to a preferred embodiment, it will be understood by those skilled in the art that various changes may be made and equivalents may be substituted without departing from the broader aspects of the invention.

I claim:

1. A dietary method of treating arthritis for a person consuming sources of nutrition including non-offending sources of nutrition and offending sources of nutrition, said offending sources of nutrition being one of either purposefully fermented sources, purposefully acidified sources and both purposefully fermented and purposefully acidified sources, said dietary method of treating arthritis comprising the steps of:
identifying pre-treatment offending sources of nutrition consumed by the person, each of said pre-treatment offending sources being one of either the purposefully fermented sources, the purposefully acidified sources, and both the purposefully fermented and the purposefully acidified sources;
compiling a list of listed pre-treatment offending sources of nutrition for the person;
determining a pre-treatment base level for said listed pre-treatment offending sources of nutrition consumed by the person during a pre-treatment consumption time period;
altering said listed pre-treatment offending sources of nutrition for the person to treatment sources of nutrition;
identifying treatment offending sources of nutrition consumed by the person, each of said treatment offending sources being one of either the purposefully fermented sources, the purposefully acidified sources, and both the purposefully fermented and the purposefully acidified sources;
compiling a list of listed treatment offending sources of nutrition for the person;
determining a treatment base level for said listed offending sources of nutrition consumed by the person during a treatment consumption time period, said treatment base level being at least fifty percent less than said pre-treatment base level; and,
maintaining consumption of said treatment offending sources of nutrition by the person at or below said treatment base level.

2. The dietary method of treating arthritis of claim 1, and wherein
a) said step of determining said pre-treatment base level includes the steps of:
designating a strength factor for each of said listed pre-treatment offending sources of nutrition;
determining a pre-treatment average periodic consumption amount for each of said listed pre-treatment offending source of nutrition consumed by the person during said pre-treatment consumption time period;
determining a pre-treatment weighted value for each of said listed pre-treatment offending source, each said pre-treatment weighted value being obtained by multiplying said pre-treatment average periodic consumption amount and said strength factor for said listed pre-treatment offending source; and,
adding said pre-treatment weighted values for each of said listed pre-treatment offending sources for the person to obtain said pre-treatment base level; and,
b) said step of determining said treatment base level includes the steps of:
designating a strength factor for each of said listed treatment offending sources;
determining a treatment average periodic consumption amount for each of said listed treatment offending sources consumed by the person during said treatment consumption time period;
determining a treatment weighted value for each of said listed treatment offending source, each said treatment weighted value being obtained by multiplying said treatment average periodic consumption amount and said strength factor for said listed treatment offending source for the person; and,
adding said treatment weighted values for each of said listed treatment offending sources for the person to obtain said treatment base level.

3. The dietary method of treating arthritis of claim 2, and wherein each of said strength factors is taken from a group of tiered strength factors.

4. The dietary method of treating arthritis of claim 2, and wherein, and said consumption time periods are each at least one week.

5. The dietary method of treating arthritis of claim 1, and wherein said pre-treatment base level is a pre-treatment base level numerical value and said treatment base level is a treatment base level numerical value, and said treatment base level numerical value is at or below 35 percent of said pre-treatment base level numerical value.

6. The dietary method of treating arthritis of claim 5, and wherein said treatment base level numerical value is at or below 20 percent of said pre-treatment base level numerical value.

7. The dietary method of treating arthritis of claim 5, and wherein said treatment base level numerical value is at or below 5 percent of said pre-treatment base level numerical value.

8. The dietary method of treating arthritis of claim 1, and wherein said treatment base level is a first lower tier level.

9. The dietary method of treating arthritis of claim 8, and further comprising the steps of:
monitoring a quantifiably determined level of arthritis pain and discomfort of the person when at or below said first lower tier level for a monitoring time period;
altering consumption of said treatment offending sources of nutrition for the person to at or below a second lower tier level when said quantifiably determined level of arthritis pain and discomfort of the person remains above a desired quantifiable level of arthritis pain and discomfort for the person; and,
maintaining consumption of said listed treatment offending sources of nutrition for the person to at or below said second lower tier level.

10. The dietary method of treating arthritis of claim 9, and further comprising the steps of:
monitoring said quantifiably determined level of arthritis pain and discomfort of the person when the person reduces consumption of said offending sources to at or below said second lower tier level of consumption of offending sources;
further altering consumption of said treatment offending sources of nutrition for the person to at or below a third lower tier level when said quantifiably determined level of arthritis pain and discomfort for the person remains above said desired quantifiable level of arthritis pain and discomfort for the person; and,
maintaining consumption of said listed treatment offending sources of nutrition for the person to at or below said third lower tier level.

11. The dietary method of treating arthritis of claim 2, and wherein said altering said pre-treatment offending sources of nutrition step includes:
reducing consumption of a first portion of said listed pre-treatment offending sources; and
replacing consumption of a second portion of said listed pre-treatment offending sources with alternative sources of nutrition.

12. The dietary method of treating arthritis of claim 11, and wherein each of said alternative sources of nutrition is one of either:
a substitute non-offending source of nutrition, and
a substitute offending source of nutrition having a lower strength factor than said strength factor for said listed pre-treatment offending source of nutrition.

13. The dietary method of treating arthritis of claim 1, and wherein said purposefully fermented sources are one of either:
a) deliberately fermented for more than one hour using one of either cultured microorganisms, cultured yeast and cultured bacteria; and,
b) deliberately fermented for more than one day using one of either wild microorganisms, wild yeast and wild bacteria.

14. The dietary method of treating arthritis of claim 1, and wherein each of the purposefully fermented sources is from a purposefully fermented group including fermented bean foods, fermented grain foods, fermented vegetable foods, fermented fruit foods, fermented honey foods, fermented dairy foods, fermented fish foods, fermented meat foods and fermented tea foods.

15. The dietary method of treating arthritis of claim 14, and wherein each of said fermented bean foods is from a bean group including cheonggukjang, doenjang, fermented bean curd, miso, natto, soy sauce, stinky tofu, tempeh, oncom, soybean paste, Beijing mung bean milk, kinama and iru;
each of said fermented grain foods is from a grain group including batter made from rice and lentil (Vigna mungo), amazake, beer, bread, choujiu, gamju, injera, kvass, makgeolli, murri, ogi, rejuvelac, sake, sikhye, sourdough, sowans, rice wine, malt whisky, grain whisky, idli, dosa, Bangla vodka, boza and chichi;
each of said fermented vegetable foods is from a vegetable group including kimchi, pickles, sauerkraut, Indian pickle, gundruk, and tursu, among others;
each of said fermented fruit foods is from a fruit group including wine, vinegar, cider, perry, brandy, atchara, nata de coco, burong mangga, asinan, vişinată, chocolate and raki;
each of said fermented honey foods is from a honey group including mead and metheglin;
each of said fermented dairy foods is from a dairy group including cheese, kefir, ryazhenka, buttermilk, kumis, shubat, quark, filmjölk, crème fraîche, smetana, skyr and yogurt;
each of said fermented fish foods is from a fish group including bagoong, faseekh, fish sauce, Garum, Hákarl, jeotgal, rakfisk, shrimp paste, surströmming and shidal;
each of said fermented meat foods is from a meat group including chin som mok, chorizo, salami, sucuk, pepperoni, nem chua, som moo and saucisson; and,
each of said fermented tea foods is from a tea group including pu-erh tea and kombucha.

16. The dietary method of treating arthritis of claim 1, and wherein the purposefully acidified sources are deliberately acidified using an acidifying agent.

17. The dietary method of treating arthritis of claim 16, and wherein said acidifying agent is from an acidifying agent group including citric acid, fumaric acid, lactic acid, acetic acid, ascorbic acid, folic acid, malic acid, sorbic acid, erythorbic acid, stearic acid, tartaric acid and phosphoric acid, and diluted solutions thereof.

18. The dietary method of treating arthritis of claim 17, and wherein each of the purposefully acidified sources has a pH level, and said acidifying agent reduces said pH level of each of the purposefully acidified sources to below a pH level of about 5.

19. The dietary method of treating arthritis of claim 1, and wherein the purposefully acidified sources include naturally acidic sources.

20. The dietary method of treating arthritis of claim 1, and wherein the arthritis is one of either osteoarthritis, rheumatoid arthritis and gout.

21. The dietary method of treating arthritis of claim 1, and wherein said method is implemented via electronic means, such as computers, laptops, smart phones, tables, smart watches, smart kitchen devices, electronic diaries, smart speakers, smart television sets, and software which collects consumption data of the patient, identifies pre-treatment offending sources of nutrition for the person, compiles a list of listed pre-treatment offending sources of nutrition for the person, determines a pre-treatment base level of offending sources of nutrition for the person, alters the pre-treatment offending sources of nutrition for the person, compiles a list of listed treatment offending sources of nutrition for the person, determines a treatment base level of offending sources for the person, monitors and helps maintaining consumption of treatment offending sources at or below treatment base level for the person or at or below lower tier levels, documents and monitors the level of pain and discomfort of the person, and alters or helps alter consumption of said treatment offending sources in lower tier levels.

22. The dietary method of treating arthritis of claim 1, and wherein the person is suffering from neck pain and said dietary method is used to treat the neck pain.

23. The dietary method of treating arthritis of claim 1, and wherein the person is suffering from back pain and said dietary method is used to treat the back pain.

24. A dietary method of treating arthritis for a person consuming sources of nutrition including non-offending sources of nutrition and purposefully fermented sources of nutrition, said dietary method of treating arthritis comprising the steps of:
identifying pre-treatment purposefully fermented sources of nutrition consumed by the person;
compiling a list of listed pre-treatment purposefully fermented sources of nutrition for the person;
determining a pre-treatment base level for said listed pre-treatment purposefully fermented sources of nutrition consumed by the person during a pre-treatment consumption time period;
altering said pre-treatment purposefully fermented sources of nutrition for the person to treatment sources of nutrition for the person;
identifying treatment purposefully fermented sources of nutrition consumed by the person;
compiling a list of listed treatment purposefully fermented sources of nutrition for the person;
determining a treatment base level for said listed treatment purposefully fermented sources of nutrition consumed by the person during a treatment consumption time period, said treatment base level being at least fifty percent less than said pre-treatment base level; and,
maintaining consumption of said treatment purposefully fermented sources of nutrition by the person at or below said treatment base level.

25. The dietary method of treating arthritis of claim 24, and wherein
a) said step of determining said pre-treatment base level includes the steps of:
designating a strength factor for each of said listed pre-treatment purposefully fermented sources of nutrition;
determining a pre-treatment average periodic consumption amount for each of said listed pre-treatment purposefully fermented sources of nutrition consumed by the person during said pre-treatment consumption time period;
determining a pre-treatment weighted value for each of said listed pre-treatment purposefully fermented sources of nutrition, each said pre-treatment weighted value being obtained by multiplying said pre-treatment average periodic consumption amount and said strength factor for said listed pre-treatment purposefully fermented source of nutrition; and,
adding said pre-treatment weighted values for each of said listed pre-treatment purposefully fermented sources of nutrition for the person to obtain said pre-treatment base level; and,
b) said step of determining said treatment base level includes the steps of:
designating a strength factor for each of said listed treatment purposefully fermented sources of nutrition;
determining a treatment average periodic consumption amount for each of said listed treatment purposefully fermented sources of nutrition consumed by the person during said treatment consumption time period;
determining a treatment weighted value for each of said listed treatment purposefully fermented sources of nutrition, each said treatment weighted value being obtained by multiplying said treatment consumption amount and said strength factor for said listed treatment purposefully fermented source of nutrition for the person; and,
adding said weighted values for each of said treatment purposefully fermented sources of nutrition for the person to obtain said treatment base level.

26. The dietary method of treating arthritis of claim 25, and wherein each of said strength factors is taken from a group of tiered strength factors.

27. The dietary method of treating arthritis of claim 25, and wherein, and said consumption time periods are each at least one week.

28. The dietary method of treating arthritis of claim 24, and wherein said pre-treatment base level is a pre-treatment base level numerical value and said treatment base level is a treatment base level numerical value, and said treatment base level numerical value is at or below 35 percent of said pre-treatment base level numerical value.

29. The dietary method of treating arthritis of claim 28, and wherein said treatment base level numerical value is at or below 20 percent of said pre-treatment base level numerical value.

30. The dietary method of treating arthritis of claim 28, and wherein said treatment base level numerical value is at or below 5 percent of said pre-treatment base level numerical value.

31. The dietary method of treating arthritis of claim 24, and wherein said treatment base level is a first lower tier level.

32. The dietary method of treating arthritis of claim 31, and further comprising the steps of:
monitoring a quantifiably determined level of arthritis pain and discomfort of the person when at or below said first lower tier level for a monitoring time period;
altering consumption of said treatment purposefully fermented sources of nutrition for the person to at or below a second lower tier level when said quantifiably determined level of arthritis pain and discomfort of the person remains above a desired quantifiable level of arthritis pain and discomfort for the person; and,
maintaining consumption of said purposefully fermented sources of nutrition for the person to at or below said second lower tier level.

33. The dietary method of treating arthritis of claim 32, and further comprising the steps of:
monitoring said quantifiably determined level of arthritis pain and discomfort of the person when the person reduces consumption of said purposefully fermented sources of nutrition to at or below said second lower tier level of consumption of purposefully fermented sources of nutrition;
further altering consumption of said treatment purposefully fermented sources of nutrition for the person to at or below a third lower tier level when said quantifiably determined level of arthritis pain and discomfort for the person remains above said desired quantifiable level of arthritis pain and discomfort for the person; and,
maintaining consumption of said purposefully fermented sources for the person to at or below said third lower tier level.

34. The dietary method of treating arthritis of claim 25, and wherein said altering said pre-treatment sources of nutrition step includes:
reducing consumption of a first portion of said listed pre-treatment purposefully fermented sources; and
replacing consumption of a second portion of said listed pre-treatment purposefully fermented sources with alternative sources of nutrition.

35. The dietary method of treating arthritis of claim 34, and wherein each of said alternative sources of nutrition is one of either:
a substitute non-offending source of nutrition, and
a substitute purposefully fermented source of nutrition having a lower strength factor than said strength factor for said listed pre-treatment purposefully fermented source of nutrition.

36. The dietary method of treating arthritis of claim 24, and wherein said purposefully fermented sources are one of either:
a) deliberately fermented for more than one hour using one of either cultured microorganisms, cultured yeast and cultured bacteria; and,
b) deliberately fermented for more than one day using one of either wild microorganisms, wild yeast and wild bacteria.

37. The dietary method of treating arthritis of claim 36, and wherein each of the purposefully fermented sources is from a purposefully fermented group including fermented bean foods, fermented grain foods, fermented vegetable foods, fermented fruit foods, fermented honey foods, fermented dairy foods, fermented fish foods, fermented meat foods and fermented tea foods.

38. The dietary method of treating arthritis of claim 37, and wherein each of said fermented bean foods is from a bean group including cheonggukjang, doenjang, fermented bean curd, miso, natto, soy sauce, stinky tofu, tempeh, oncom, soybean paste, Beijing mung bean milk, kinama and iru;
each of said fermented grain foods is from a grain group including batter made from rice and lentil (Vigna mungo), amazake, beer, bread, choujiu, gamju, injera, kvass, makgeolli, murri, ogi, rejuvelac, sake, sikhye, sourdough, sowans, rice wine, malt whisky, grain whisky, idli, dosa, Bangla vodka, boza and chichi;
each of said fermented vegetable foods is from a vegetable group including kimchi, pickles, sauerkraut, Indian pickle, gundruk, and tursu, among others;
each of said fermented fruit foods is from a fruit group including wine, vinegar, cider, perry, brandy, atchara, nata de coco, burong mangga, asinan, vişinată, chocolate and raki;
each of said fermented honey foods is from a honey group including mead and metheglin;
each of said fermented dairy foods is from a dairy group including cheese, kefir, ryazhenka, buttermilk, kumis, shubat, quark, filmjölk, crème fraîche, smetana, skyr and yogurt;
each of said fermented fish foods is from a fish group including bagoong, faseekh, fish sauce, Garum, Hákarl, jeotgal, rakfisk, shrimp paste, surströmming and shidal;
each of said fermented meat foods is from a meat group including chin som mok, chorizo, salami, sucuk, pepperoni, nem chua, som moo and saucisson; and,
each of said fermented tea foods is from a tea group including pu-erh tea and kombucha.

39. The dietary method of treating arthritis of claim 24, and wherein the arthritis is one of either osteoarthritis, rheumatoid arthritis and gout.

40. The dietary method of treating arthritis of claim 24, and wherein said method is implemented via electronic means, such as computers, laptops, smart phones, tables, smart watches, smart kitchen devices, electronic diaries, smart speakers, smart television sets, and software which collects consumption data of the patient, identifies pre-treatment offending sources of nutrition for the person, compiles a list of listed pre-treatment offending sources of nutrition for the person, determines a pre-treatment base level of offending sources of nutrition for the person, alters the pre-treatment offending sources of nutrition for the person, compiles a list of listed treatment offending sources of nutrition for the person, determines a treatment base level of offending sources for the person, monitors and helps maintaining consumption of treatment offending sources at or below treatment base level for the person or at or below lower tier levels, documents and monitors the level of pain and discomfort of the person, and alters or helps alter consumption of said treatment offending sources in lower tier levels.

41. The dietary method of treating arthritis of claim 24, and wherein the person is suffering from neck pain and said dietary method is used to treat the neck pain.

42. The dietary method of treating arthritis of claim 24, and wherein the person is suffering from back pain and said dietary method is used to treat the back pain.

43. A dietary method of treating arthritis for a person consuming sources of nutrition including non-offending sources of nutrition and purposefully acidified sources of nutrition, said dietary method of treating arthritis comprising the steps of:
identifying pre-treatment purposefully acidified sources of nutrition consumed by the person;
compiling a list of listed pre-treatment purposefully acidified sources of nutrition for the person;
determining a pre-treatment base level of purposefully acidified sources of nutrition for the person during a pre-treatment consumption time period;
altering said listed pre-treatment purposefully acidified sources of nutrition for the person to treatment sources of nutrition;
identifying treatment purposefully acidified sources of nutrition consumed by the person;
compiling a list of listed treatment purposefully acidified sources of nutrition for the person;
determining a treatment base level of purposefully acidified sources of nutrition consumed by the person during a treatment consumption time period, said treatment base level being at least fifty percent less than said pre-treatment base level; and,
maintaining consumption of said treatment purposefully acidified sources of nutrition by the person at or below said treatment base level.

44. The dietary method of treating arthritis of claim 43, and wherein
a) said step of determining said pre-treatment base level includes the steps of:
designating a strength factor for each of said listed pre-treatment purposefully acidified sources of nutrition;
determining a pre-treatment average periodic consumption amount for each of the listed pre-treatment purposefully acidified sources of nutrition consumed by the person during said pre-treatment consumption time period;
determining a pre-treatment weighted value for each of said listed pre-treatment purposefully acidified source of nutrition, each said pre-treatment weighted value being obtained by multiplying said pre-treatment average periodic consumption amount and said strength factor for said listed pre-treatment purposefully acidified source of nutrition; and,
adding said weighted values for each of said listed pre-treatment purposefully acidified sources of nutrition for the person to obtain said pre-treatment base level; and,
b) said step of determining said treatment base level includes the steps of:
designating a strength factor for each of said listed treatment purposefully acidified sources of nutrition;
determining a treatment average periodic consumption amount for each of said listed treatment purposefully acidified sources of nutrition consumed by the person during said treatment consumption time period;
determining a treatment weighted value for each of said listed treatment purposefully acidified source of nutrition, each said treatment weighted value being obtained by multiplying said treatment average periodic consumption amount and said strength factor for said listed treatment purposefully acidified source of nutrition for the person; and,
adding said weighted values for each of said listed treatment purposefully acidified sources of nutrition for the person to obtain said treatment base level.

45. The dietary method of treating arthritis of claim 44, and wherein each of said strength factors is taken from a group of tiered strength factors.

46. The dietary method of treating arthritis of claim 44, and wherein, and said consumption time periods are each at least one week.

47. The dietary method of treating arthritis of claim 43, and wherein said pre-treatment base level is a pre-treatment base level numerical value and said treatment base level is a treatment base level numerical value, and said treatment base level numerical value is at or below 35 percent of said pre-treatment base level numerical value.

48. The dietary method of treating arthritis of claim 47, and wherein said treatment base level numerical value is at or below 20 percent of said pre-treatment base level numerical value.

49. The dietary method of treating arthritis of claim 47, and wherein said treatment base level numerical value is at or below 5 percent of said pre-treatment base level numerical value.

50. The dietary method of treating arthritis of claim 43, and wherein said treatment base level is a first lower tier level.

51. The dietary method of treating arthritis of claim 50, and further comprising the steps of:
monitoring a quantifiably determined level of arthritis pain and discomfort of the person when at or below said first lower tier level for a monitoring time period;
altering consumption of said treatment purposefully acidified sources of nutrition for the person to at or below a second lower tier level when said quantifiably determined level of arthritis pain and discomfort of the person remains above a desired quantifiable level of arthritis pain and discomfort for the person; and,
maintaining consumption of said purposefully acidified sources of nutrition for the person to at or below said second lower tier level.

52. The dietary method of treating arthritis of claim 51, and further comprising the steps of:
monitoring said quantifiably determined level of arthritis pain and discomfort of the person when the person reduces consumption of said purposefully acidified sources of nutrition to at or below said second lower tier level of consumption of purposefully acidified sources of nutrition;
further altering consumption of said treatment purposefully acidified sources of nutrition for the person to at or below a third lower tier level when said quantifiably determined level of arthritis pain and discomfort for the person remains above said desired quantifiable level of arthritis pain and discomfort for the person; and,
maintaining consumption of said purposefully acidified sources for the person to at or below said third lower tier level.

53. The dietary method of treating arthritis of claim 44, and wherein said altering said pre-treatment sources of nutrition step includes:
reducing consumption of a first portion of the purposefully acidified sources; and
replacing consumption of a second portion of the purposefully acidified sources with alternative sources of nutrition.

54. The dietary method of treating arthritis of claim 53, and wherein each of said alternative sources of nutrition is one of either:
a substitute non-offending source of nutrition, and
a substitute purposefully acidified source of nutrition having a lower strength factor than said strength factor for said listed pre-treatment purposefully acidified source of nutrition.

55. The dietary method of treating arthritis of claim 43, and wherein the purposefully acidified sources are deliberately acidified using an acidifying agent.

56. The dietary method of treating arthritis of claim 55, and wherein said acidifying agent is from the group including citric acid, fumaric acid, lactic acid, acetic acid, ascorbic acid, folic acid, malic acid, sorbic acid, erythorbic acid, stearic acid, tartaric acid and phosphoric acid, and diluted solutions thereof.

57. The dietary method of treating arthritis of claim 56, and wherein each of the purposefully acidified sources has a pH level, and said acidifying agent reduces said pH level of each of the purposefully acidified sources to below a pH level of about 5.

58. The dietary method of treating arthritis of claim 43, and wherein the purposefully acidified sources include naturally acidic sources.

59. The dietary method of treating arthritis of claim 43, and wherein the arthritis is one of either osteoarthritis, rheumatoid arthritis and gout.

60. The dietary method of treating arthritis of claim 43, and wherein said method is implemented via electronic means, such as computers, laptops, smart phones, tables, smart watches, smart kitchen devices, electronic diaries, smart speakers, smart television sets, and software which collects consumption data of the patient, identifies pre-treatment offending sources of nutrition for the person, compiles a list of listed pre-treatment offending sources of nutrition for the person, determines a pre-treatment base level of offending sources of nutrition for the person, alters the pre-treatment offending sources of nutrition for the person, compiles a list of listed treatment offending sources of nutrition for the person, determines a treatment base level of offending sources for the person, monitors and helps maintaining consumption of treatment offending sources at or below treatment base level for the person or at or below lower tier levels, documents and monitors the level of pain and discomfort of the person, and alters or helps alter consumption of said treatment offending sources in lower tier levels.

61. The dietary method of treating arthritis of claim 43, and wherein the person is suffering from neck pain and said dietary method is used to treat the neck pain.

62. The dietary method of treating arthritis of claim 43, and wherein the person is suffering from back pain and said dietary method is used to treat the back pain.
